(19) United States
(12) Patent Application Publication
(10) Pub. No.: US 2007/0274994 A1
(43) Pub. Date: Nov. 29, 2007

(54) Virulence-Associated Adhesins

(75) Inventors: Vega Masignani, Siena (IT); Beatrice Maria Arico, Siena (IT)

Correspondence Address: NOVARTIS VACCINES AND DIAGNOSTICS INC., INTELLECTUAL PROPERTY R338, P.O. BOX 8097, Emeryville, CA, 94662-8097, US

(73) Assignee: Novartis Vaccines and Diagnostics, Inc., Emeryville, CA (US)

(21) Appl. No.: 10/562,191

(22) PCT Filed: Jun. 25, 2004
(86) PCT No.: PCT/IB04/02351

§ 371 Date: Oct. 26, 2006

(30) Foreign Application Priority Data
Jun. 26, 2003 (GB) 0315022.4

(51) Int. Cl. A61K 39/395; A61K 38/00; A61K 48/00; C07K 14/00; C07K 16/18; C07H 21/00; A61K 39/00

(52) U.S. Cl. 424/139.1; 424/204.1; 514/12; 514/44; 530/350; 530/387.9; 536/23.53; 536/23.72

(57) ABSTRACT

Virulence-associated antigens involved in adhesin have been identified in several organisms: Haemophilus influenzae biogroup aegyptius; Escherichia coli K1; EHEC E.coli; Actinobacillus actinomycetencomitans; Haemophilus somnus; Haemophilus ducreyi; EPEC E.coli; EA EC E.coli; uropathogenic E.coli; Shigella flexneri; Brucella melitensis; Brucella suis; Ralstonia solanacearum; Sinorhizobium meliloti; Bradorhizobium japonicum; and Burkholderia fungorum. Although the degree of sequence identity between the adhesins is low, they share a common arrangement of domains from N-terminus to C-terminus, namely: a leader peptide; a globular head; a coiled-coil region; and a transmembrane anchor region.

All documents cited herein are incorporated by reference in their entirety.

### TECHNICAL FIELD

This invention is in the field of bacterial adhesin. In particular, it relates to virulence-related adhesin antigens derived from Haemophilus influenzae, Escherichia coli and other organisms.

### BACKGROUND ART

The Gram negative Haemophilus genus includes H.influenzae, H.aegyptius (also referred to as H. influenzae biogroup aegyptius), H.decreyi and H.somnus. These bacteria can cause diseases including conjunctivitis, chancroid, purpuric fever, meningitis, pneumonia and epiglottitis. H.influenzae is the most commonly-found pathogen in this genus, and includes both typeable (encapsulated) and non-typeable (non-capsulated; ‘NTHi’) strains.

A vaccine against H influenzae type B (‘Hib’) based on a conjugate of its capsular saccharide and a carrier protein has been enormously successful, but there has been little progress in providing protection against other members of the species. In particular, type D H.influenzae and non-typeable H.influenzae remain problematic.

Similarly, vaccines remain unavailable for other bacterial pathogens such as enterotoxigenic (ETEC), enteropathogenic (EPEC), enteroaggregative (EAEC), enterohemorrhagic (EHEC) and shiga-toxic (STEC) strains of Escherichia coli.

It is an object of the invention to provide materials and methods to improve the prevention and treatment of infections caused by such bacteria. More particularly, it is an object of the invention to provide materials suitable for immunising against bacterial infections.

### DISCLOSURE OF THE INVENTION

Virulence-associated antigens involved in adhesin have been identified in several bacteria and other organisms, and these antigens are useful for the diagnosis, prevention and treatment of bacterial infections (particularly those caused by virulent strains). In particular, antigens have been identified in: Haemophilus influenzae biogroup aegyptius (SEQ ID NO: 1); Escherichia coli K1 (SEQ ID NO^{S}: 2 & 3) and also in EHEC strain EDL933; Actinobacillus actinomycetemcomitans (SEQ ID NO: 4); Haemophilus somnus (SEQ ID NO: 5); Haemophilus ducreyi (SEQ ID NO: 6); EPEC E.coli strain E2348/69 (SEQ ID NO: 7); EPEC (SEQ ID NO: 18); EAEC E.coli strain O42 (SEQ ID NO^{S}: 8 & 9); uropathogenic E.coli (SEQ ID NO: 10); Shigella flexneri (SEQ ID NO: 11); Brucella melitensis (SEQ ID NO: 12); Brucella suis (SEQ ED NO: 13); Ralstonia solanacearum (SEQ ID NO: 14); Sinorhizobium meliloti (SEQ ID NO: 15); Bradorhizobium japonicum (SEQ ID NO: 16); and Burkholderia fungorum (SEQ ID NO: 17).

Although the degree of sequence identity between the antigens of the invention is low, an appreciation of the antigens at a level beyond simple primary sequence information shows that they share a common arrangement of domains from N-terminus to C-terminus, namely:
a leader peptide
a globular head
a coiled-coil region
a transmembrane anchor region

Sequence similarity between the various antigens is largely restricted to the C-terminal anchor region. This arrangement of domains is shared with N.meningitidis protein NadA {1}.

The positions of these features in SEQ ID NO^{S}: 1 to 18 are as follows:

| SEQ ID | Organism | Length | Leader | Head | Coiled-coil | Anchor |
|---|---|---|---|---|---|---|
| 1 | H. aegyptius | >223 | 1-26 | 27-55 | 56-184 | 185 . . . |
| 2 | EHEC | 338 | 1-23 | 24-207 | 208-266 | 267-338 |
| 3 | | 1588 | 1-53 | 54-1515 * | | 1516-1588 |
| 4 | A. actinomycetemcomitans | 295 | 1-25 | 26-150 | 151-222 | 223-295 |
| 5 | H. somnus | 452 | 1-26 | 27-158 | 159-378 | 379-452 |
| 6 | H. ducreyi | 273 | 1-21 | 22-198 * | | 199-273 |
| 7 | EPEC | 338 | 1-24 | 25-209 | 210-266 | 267-338 |
| 8 | EAEC | 717 | 1-23 | 24-109 | 110-645 | 646-717 |
| 9 | | 1743 | 1-53 | 54-1670 * | | 1671-1743 |
| 10 | UPEC | 1778 | 1-53 | 54-1705 * | | 1706-1778 |
| 11 | S. flexneri | 990 | 1-917 * | | | 918-990 |
| 12 | B. melitensis | 227 | 1-27 | 28-122 | 123-154 | 155-227 |
| 13 | B. suis | 311 | 1-27 | 28-206 | 207-238 | 239-311 |
| 14 | R. solanacearum | 1309 | 1-230 * | | 231-708 | 1239-1309 |
| 15 | S. meliloti | 1291 | 1-1219 * | | | 1220-1291 |
| 16 | B. japonicum | 372 | 1-72 | 73-300 * | | 301-372 |
| 17 | B. fungorum | 3399 | 1-57 | 58-3328 * | | 3329-3399 |
| 18 | EPEC | 577 | 1-504 * | | | 505-577 |
| 51 | H. aegyptius | 256 | 1-26 | 27-55 | 56-184 | 185-256 |

* The boundary between domains is less distinct for some polypeptides of the invention

Antigens

The invention provides a polypeptide comprising one or more of the following amino acid sequences: any of SEQ ID NO^{S}: 1 to 18, SEQ ID NO: 51, and SEQ ID NO: 54.

The invention also provides a polypeptide comprising an amino acid sequence: (a) having at least m % identity to one or more of SEQ ID NO^{S}: 1-18, 51 & 54, where m is 50 or more (e.g. 60, 65, 70, 75, 80, 85, 90, 91, 92, 93, 94, 95, 96, 97, 98, 99, 99.5 or more); and/or (b) which is a fragment of at least n consecutive amino acids of one or more of SEQ ID NO^{S}: 1-18, 51 & 54, wherein n is 7 or more (e.g. 8, 10, 12, 14, 16, 18, 20, 25, 30, 35, 40, 50, 60, 70, 80, 90, 100, 150, 200, 250 or more). These polypeptides include variants (e.g. allelic variants, homologs, orthologs, paralogs, mutants, etc.) of SEQ ID NO^{S}: 1-18, 51 & 54.

Preferred fragments of (b) comprise an epitope from one or more of SEQ ID NO^{S}: 1-18, 51 & 54, preferably a B-cell epitope. B-cell epitopes can be identified empirically or can be predicted algorithmically.

Other preferred fragments of (b) lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25, 45 or more) from the N-terminus of the relevant amino acid sequence from SEQ ID NO^{S}: 1-18, 51 & 54. In particular, preferred fragments omit at least the N-terminus leader sequence (and the omitted leader sequence may be replaced by a heterologous leader sequence).

Other preferred fragments omit one or more (i.e. 1, 2, or 3) of the four domains of SEQ ID NO^{S}: 1-18 & 51, based on the above table. Other preferred fragments consist of one or more (i.e. 1, 2, or 3) of the four domains of SEQ ID NO^{S}: 1-18 & 51.

Preferred polypeptides of the invention are presented in oligomeric form (e.g. dimers, trimers, tetramers, etc.). Trimers are preferred, but monomeric polypeptides of the invention are also useful.

The invention also provides polypeptides of the formula NH₂-A-{-X-L-})ₓ-B—COOH, wherein:
X comprises an amino acid sequence: (a) having at least m % identity to one or more of SEQ ID NO^{S}: 1-18, 51 & 54; and/or (b) which is a fragment of at least n consecutive amino acids of one or more of SEQ ID NO^{S}: 1-18, 51 & 54, as defined above;
L is an optional linker amino acid sequence;
A is an optional N-terminal amino acid sequence;
B is an optional C-terminal amino acid sequence; and
x is 1, 2, 3, 4, 5, 6, 7, 8, 9, 10, 11, 12, 13, 14, 15, 16, 17 or 18 (preferably x=2).

Where a —X— moiety has a leader peptide, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₓ will be omitted. This is equivalent to deleting all leader peptides and using the leader peptide of X₁ as moiety -A-.

For each x instances of {—X-L-}, —X— may be the same or different, and linker amino acid sequence -L- may be present or absent. For instance, when x=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g. 20 or fewer amino acids i.e. 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. A useful linker is GSGGGG (SEQ ID NO: 19), with the Gly-Ser dipeptide being formed from a BamHI restriction site, thus aiding cloning and manipulation, and the (Gly)₄ tetrapeptide being a typical poly-glycine linker.

-A- is an optional N-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. HiSₕ where h=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₕ where h=3, 4, 5, 6, 7, 8, 9, 10 or more), or sequences which enhance protein stability. Other suitable C-terminal amino acid sequences will be apparent to those skilled in the art.

The invention also provides polypeptides comprising the amino acid sequence:
-A-W₁—W₂—W₃—W₄—B—
wherein:
A is an optional sequence as defined above (preferably at the N-terminus of the polypeptide);
B is an optional sequence as defined above (preferably at the C-terminus of the polypeptide);
W₁ is an optional amino acid sequence: (a) having at least m % identity to the leader peptide of one or more of SEQ ID NO^{S}: 1-18 & 51; and/or (b) which is a fragment of at least n consecutive amino acids of the leader peptide of one or more of SEQ ID NO^{S}: 1-18 & 51;
W₂ is an optional amino acid sequence: (a) having at least m % identity to the globular head domain of one or more of SEQ ID NO^{S}: 1-18 & 51; and/or (b) which is a fragment of at least n consecutive amino acids of the globular head domain of one or more of SEQ ID NO^{S}: 1-18, & 51;
W₃ is an optional amino acid sequence: (a) having at least m % identity to the coiled-coil domain of one or more of SEQ ID NO^{S}: 1-18 & 51; and/or (b) which is a fragment of at least n consecutive amino acids of the coiled-coil domain of one or more of SEQ ID NO^{S}: 1-18 & 51;
W₄ is an optional amino acid sequence: (a) having at least m % identity to the transmembrane anchor region of one or more of SEQ ID NO^{S}: 1-18 & 51; and/or (b) which is a fragment of at least n consecutive amino acids of the transmembrane anchor region of one or more of SEQ ID NO^{S}: 1-18 & 51;

provided that at least one of W₁, W₂, W₃ or W₄ is present.

The invention also provides a polypeptide comprising a polypeptide as described above, wherein the amino acid sequence of the polypeptide contains one or more amino acid mutations. The mutation(s) preferably result in the reduction or removal of an activity of a polypeptide of the invention which is responsible directly or indirectly for virulence or adhesin. For example, the mutation may inhibit an enzymatic activity or may remove a binding site in the protein. Mutation may involve deletion, substitution, and/or insertion, any of which may be involve one or more amino acids. As an alternative, the mutation may involve truncation.

Mutagenesis of virulence factors is a well-established science for many bacteria {e.g. toxin mutagenesis described in refs. 2 to 8}. Mutagenesis may be specifically targeted to nucleic acid encoding a polypeptide of the invention. Alternatively, mutagenesis may be global or random (e.g. by irradiation, chemical mutagenesis, etc.), which will typically be followed by screening bacteria for those in which a mutation has been introduced into a gene encoding a polypeptide of the invention. Such screening may be by hybridisation assays (e.g. Southern or Northern blots etc.), primer-based amplification (e.g. PCR), sequencing, proteomics, aberrant SDS-PAGE gel migration, etc.

Polypeptides of the invention can be prepared by various means (e.g. recombinant expression, purification from cell culture, chemical synthesis, etc.) and in various forms (e.g. native, fusions, non-glycosylated, lipidated, etc.). They are preferably prepared in substantially pure form (i.e. substantially free from other bacterial or host cell proteins).

Whilst expression of the polypeptides of the invention may take place in the native host, the invention preferably utilises a heterologous host. The heterologous host may be prokaryotic (e.g. a bacterium) or eukaryotic. It is preferably E.coli, but other suitable hosts include Bacillus subtilis, Vibrio cholerae, Salmonella typhi, Salmonella typhimurium, Neisseria lactamica, Neisseria cinerea, Mycobacteria (e.g. M.tuberculosis), yeasts, etc.

Where a polypeptide of the invention is related to SEQ ID NO: 51, it preferably comprises at least 224 (e.g. 224, 225, 226, 227, 228, 229, 230, 235, 240, 245, 250, 255 or more) amino acids.

The invention also provides an adhesin from Haemophilus aegyptius, wherein the adhesin comprises: (a) amino acid sequence SEQ ID NO: 52; (b) an amino acid sequence having at least m % identity to SEQ ID NO: 52; and/or (c) an amino acid sequence which is a fragment of at least n consecutive amino acids of SEQ ID NO: 52.

Antibodies

The invention also provides antibodies which bind to polypeptides of the invention.

Antibody of the invention preferably has an affinity for a polypeptide of the invention of at least 10⁻⁷ M e.g. 10⁻⁸ M, 10⁻⁹ M, 10⁻¹⁰ M or tighter. Preferred antibodies can block the ability of a polypeptide of the invention to bind to a human cell.

Antibodies of the invention may be polyclonal or monoclonal and may be produced by any suitable means (e.g. by recombinant expression, purification from cell culture, chemical synthesis, etc.) and in various forms (e.g. native, fusions, glycosylated, non-glycosylated, etc.). They are preferably prepared in substantially pure form (i.e. substantially free from other antibodies).

The term “antibody”includes whole antibodies, Fv, scFv, Fc, Fab, F(ab′)₂, etc.

Antibodies of the invention may include a label. The label may be detectable directly, such as a radioactive or fluorescent label. Alternatively, the label may be detectable indirectly, such as an enzyme whose products are detectable (e.g. luciferase, β-galactosidase, peroxidase, etc.).

Antibodies of the invention may be attached to a solid support.

Antibodies of the invention may be prepared by administering (e.g. injecting) a polypeptide of the invention to an appropriate animal (e.g. a rabbit, hamster, mouse or other rodent).

To increase compatibility with the human immune system, the antibodies may be chimeric or humanized {e.g. refs. 9 & 10}, or fully human antibodies may be used. Because humanized antibodies are far less immunogenic in humans than the original non-human monoclonal antibodies, they can be used for the treatment of humans with far less risk of anaphylaxis. Thus, these antibodies may be preferred in therapeutic applications that involve in vivo administration to a human such as, use as radiation sensitizers for the treatment of neoplastic disease or use in methods to reduce the side effects of cancer therapy.

Humanized antibodies may be achieved by a variety of methods including, for example: (1) grafting non-human complementarity determining regions (CDRs) onto a human framework and constant region (“humanizing”), with the optional transfer of one or more framework residues from the non-human antibody; (2) transplanting entire non-human variable domains, but “cloaking” them with a human-like surface by replacement of surface residues (“veneering”). In the present invention, humanized antibodies will include both “humanized” and “veneered” antibodies. {11, 12, 13, 14, 15, 16, 17). Humanized or fully-human antibodies can also be produced using transgenic animals that are engineered to contain human immunoglobulin loci.

The phrase “constant region” refers to the portion of the antibody molecule that confers effector functions. In chimeric antibodies, mouse constant regions are substituted by human constant regions.

The constant regions of humanized antibodies are derived from human immunoglobulins. The heavy chain constant region can be selected from any of the 5 isotypes: alpha, delta, epsilon, gamma or mu.

Nucleic Acids

The invention also provides nucleic acid encoding the polypeptides of the invention. Furthermore, the invention provides nucleic acid which can hybridise to this nucleic acid, preferably under “high stringency” conditions (e.g. 65° C. in a 0.1×SSC, 0.5% SDS solution).

Nucleic acid according to the invention can be prepared in many ways (e.g. by chemical synthesis, from genomic or cDNA libraries, from the organism itself, etc.) and can take various forms (e.g. single stranded, double stranded, vectors, probes, etc.). They are preferably prepared in substantially pure form (i.e. substantially free from other bacterial or host cell nucleic acids).

The term “nucleic acid” includes DNA and RNA, and also their analogues, such as those containing modified backbones (e.g. phosphorothioates, etc.), and also peptide nucleic acids (PNA), etc. The invention includes nucleic acid comprising sequences complementary to those described above (e.g. for antisense or probing purposes).

Immunogenic Compositions and Medicaments

Based on the structural and functional similarities to NadA, which is a good anti-meningococcal immunogen {1}, including their association with virulence, the polypeptides of the invention should also be useful for immunisation purposes.

The invention provides a composition comprising a polypeptide and/or a nucleic acid and/or an antibody of the invention. Compositions of the invention are preferably immunogenic compositions, and are more preferably vaccine compositions. Vaccines according to the invention may either be prophylactic (i.e. to prevent infection) or therapeutic (i.e. to treat infection), but will typically be prophylactic.

The pH of the composition is preferably between 6 and 8, preferably about 7. The pH may be maintained by the use of a buffer. The composition may be sterile and/or pyrogen-free. The composition may be isotonic with respect to humans.

The invention also provides a composition of the invention for use as a medicament. The medicament is preferably able to raise an immune response in a mammal (i.e. it is an immunogenic composition) and is more preferably a vaccine.

The invention also provides the use of one or more (e.g. 2, 3, 4, 5, 6) of the polypeptides of the invention in the manufacture of a medicament for raising an immune response in a mammal. The medicament is preferably a vaccine.

The invention also provides a method for raising an immune response in a mammal comprising the step of administering an effective amount of a composition of the invention. The immune response is preferably protective and preferably involves antibodies and/or cell-mediated immunity. The method may raise a booster response.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

These uses and methods are preferably for the prevention and/or treatment of a disease caused by Haemophilus influenzae biogroup aegyptius, Escherichia coli (particularly EHEC, EAEC, ETEC, EPEC and UPEC strains), Actinobacillus actinomycetemcomitans, Haemophilus somnus, Haemophilus ducreyi, Shigella flexneri, Brucella melitensis, Brucella suis, Ralstonia solanacearum, Sinorhizobium meliloti, Bradorhizobium japonicum and Burkholderia fungorum. Thus the invention is suitable for the prevention and/or treatment of diseases including: conjunctivitis, chancroid, purpuric fever, meningitis, pneumonia, epiglottitis, peri-implantitis, periodontal disease, gingivitis, bovine encephalitis, arthritis, myocarditis, diarrhoea, ovine abortion, orchitis, undulant fever, porcine reproductive wastage, brucellosis, etc.

One way of checking efficacy of therapeutic treatment involves monitoring bacterial infection after administration of the composition of the invention. One way of checking efficacy of prophylactic treatment involves monitoring immune responses against the polypeptides after administration of the composition.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral (e.g. tablet, spray), vaginal, topical, transdermal {e.g. see ref. 18} or transcutaneous {e.g. see refs. 19 & 20}, intranasal (e.g. see ref. 21 }, ocular, aural, pulmonary or other mucosal administration.

The invention may be used to elicit systemic and/or mucosal immunity.

Dosage treatment can be a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc.

Bacterial infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

The invention also provides the polypeptides of the invention (including NadA itself) for use as adjuvants (parenteral and/or mucosal). Similarly, the invention provides a composition comprising a polypeptide of the invention in admixture with a second antigen, whereby the polypeptide of the invention enhances the immune response against the second antigen when administered to a patient.

Further Components of the Composition

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in reference 22.

Vaccines of the invention may be administered in conjunction with other immunoregulatory agents. In particular, compositions will usually include an adjuvant. Preferred further adjuvants include, but are not limited to: (A) aluminium salts, including hydroxides (e.g. oxyhydroxides), phosphates (e.g. hydroxyphoshpates, orthophosphates), sulphates, etc. {e.g. see chapters 8 & 9 of ref. 23}), or mixtures of different aluminium compounds, with the compounds taking any suitable form (e.g. gel, crystalline, amorphous, etc.), and with adsorption being preferred; (B) MF59 (5% Squalene, 0.5% Tween 80, and 0.5% Span 85, formulated into submicron particles using a microfluidizer) {see Chapter 10 of 23; see also ref. 24}; (C) liposomes {see Chapters 13 and 14 of ref. 23}; (D) ISCOMs {see Chapter 23 of ref. 23}, which may be devoid of additional detergent {25}; (E) SAF, containing 10% Squalane, 0.4% Tween 80, 5% pluronic-block polymer L121, and thr-MDP, either microfluidized into a submicron emulsion or vortexed to generate a larger particle size emulsion {see Chapter 12 of ref. 23}; (F) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (G) saponin adjuvants, such as QuilA or QS21 {see Chapter 22 of ref. 23}, also known as Stimulon™ {26}; (H) chitosan {e.g. 27}; (I) complete Freund's adjuvant (CFA) and incomplete Freund's adjuvant (IFA); (J) cytokines, such as interleukins (e.g. IL-1, IL-2, IL-4, IL-5, IL-6, IL-7, IL-12, etc.), interferons (e.g. interferon-γ), macrophage colony stimulating factor, tumor necrosis factor, etc. {see Chapters 27 & 28 of ref 23}; (K) monophosphoryl lipid A (MPL) or 3-O-deacylated MPL (3dMPL) {e.g. chapter 21 of ref. 23}; (L) combinations of 3dMPL with, for example, QS21 and/or oil-in-water emulsions {28}; (M) a polyoxyethylene ether or a polyoxyethylene ester {29}; (N) a polyoxyethylene sorbitan ester surfactant in combination with an octoxynol {30} or a polyoxyethylene alkyl ether or ester surfactant in combination with at least one additional non-ionic surfactant such as an octoxynol {31}; (N) a particle of metal salt {32}; (O) a saponin and an oil-in-water emulsion {33}; (P) a saponin (e.g. QS21)+3dMPL+IL-12 (optionally + a sterol) {34}; (Q) E.coli heat-labile enterotoxin (“LT”), or detoxified mutants thereof, such as the K63 or R72 mutants {e.g. Chapter 5 of ref. 35}; (R) cholera toxin (“CT”), or detoxified mutants thereof {e.g. Chapter 5 of ref. 35}; (S) double-stranded RNA; (T) microparticles (i.e. a particle of ˜100 nm to ˜150 μm in diameter, more preferably —200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) being preferred, optionally treated to have a negatively-charged surface (e.g. with SDS) or a positively-charged surface (e.g. with a cationic detergent, such as CTAB); (U) oligonucleotides comprising CpG motifs i.e. containing at least one CG dinucleotide, with 5-methylcytosine optionally being used in place of cytosine; (V) monophosphoryl lipid A mimics, such as aminoalkyl glucosaminide phosphate derivatives e.g. RC-529 {36}; (W) polyphosphazene (PCPP); (X) a bioadhesive {37} such as esterified hyaluronic acid microspheres {38} or a mucoadhesive selected from the group consisting of cross-linked derivatives of poly(acrylic acid), polyvinyl alcohol, polyvinyl pyrollidone, polysaccharides and carboxymethylcellulose; or (Y) other substances that act as immunostimulating agents to enhance the effectiveness of the composition {e.g. see Chapter 7 of ref. 23}. Aluminium salts and MF59 are preferred adjuvants for parenteral immunisation. Mutant toxins are preferred mucosal adjuvants.

Muramyl peptides include N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine MTP-PE), etc.

The composition may include an antibiotic.

Further Antigens

As well as containing polypeptides of the invention, the compositions of the invention may also include one or more further antigens. Further antigens for inclusion may be, for example:
a saccharide antigen from N.meningitidis serogroup A, C, W135 and/or Y, such as the oligosaccharide disclosed in ref. 39 from serogroup C {see also ref. 40} or the oligosaccharides of ref. 41.
antigens from Helicobacter pylori such as CagA {42 to 45}, VacA {46, 47}, NAP {48, 49, 50}, HopX {e.g. 51}, HopY {e.g. 51} and/or urease.
a saccharide antigen from Streptococcus pneumoniae {e.g. 52, 53, 545}.
a protein antigen from Streptococcus pneumoniae {e.g. 55}.
an antigen from hepatitis A virus, such as inactivated virus {e.g. 56, 57}.
an antigen from hepatitis B virus, such as the surface and/or core antigens {e.g. 57, 58}.
an antigen from hepatitis C virus {e.g. 59}.
a diphtheria antigen, such as a diphtheria toxoid {e.g. chapter 3 of ref. 60} e.g. the CRM₁₉₇ mutant {e.g. 61}.
a tetanus antigen, such as a tetanus toxoid {e.g. chapter 4 of ref. 60}.
an antigen from Bordetella pertussis, such as pertussis holotoxin (PT) and filamentous haemagglutinin (FHA) from B.pertussis, optionally also in combination with pertactin and/or agglutinogens 2 and 3 {e.g. refs. 62 & 63}; whole-cell pertussis antigen may also be used.
a saccharide antigen from Haemophilus influenzae B {e.g. 40}.
polio antigen(s) {e.g. 64, 65} such as OPV or, preferably, IPV.
a protein antigen from N.meningitidis serogroup B {e.g. refs. 66 to 77}, such as NadA.
an outer-membrane vesicle (OMV) preparation from N.meningitidis serogroup B, such as those disclosed in refs. 78, 79, 80, 81, etc.
an antigen from Chlamydia pneumoniae {e.g. refs. 82 to 88}.
an antigen from Chlamydia trachomatis {e.g. 89}.
an antigen from Porphyromonas gingivalis {e.g. 90}.
rabies antigen(s) {e.g. 91} such as lyophilised inactivated virus {e.g. 92, RabAvert™}.
measles, mumps and/or rubella antigens {e.g. chapters 9, 10 & 11 of ref. 60}.
influenza antigen(s) {e.g. chapter 19 of ref. 60}, such as the hemagglutinin and/or neuraminidase surface proteins.
an antigen from N.gonorrhoeae {e.g. 93, 94, 95, 96}.
antigen(s) from a paramyxovirus such as respiratory syncytial virus (RSV {97, 98}) and/or parainfluenza virus (PIV3 {99}).
an antigen from Moraxella catarrhalis {e.g. 100}, such as UspA1 and/or UspA2
an antigen from Streptococcus pyogenes (group A streptococcus) {e.g. 101, 102, 103}.
an antigen from Streptococcus agalactiae (group B streptococcus) {e.g. 104}.
an antigen from Staphylococcus aureus {e.g. 105}.
an antigen from Bacillus anthracis {e.g. 106, 107, 108}.
an antigen from a virus in the flaviviridae family (genus flavivirus), such as from yellow fever virus, Japanese encephalitis virus, four serotypes of Dengue viruses, tick-borne encephalitis virus, West Nile virus.
an antigen from Pseudomonas.
an antigen from a HIV e.g. a HIV-1 or HIV-2.
an antigen from a rotavirus.
a pestivirus antigen, such as from classical porcine fever virus, bovine viral diarrhoea virus, and/or border disease virus.
a parvovirus antigen e.g. from parvovirus B19.
a coronavirus antigen e.g. from the SARS coronoavirus.
a cancer antigen, such as those listed in Table 1 of ref. 109 or in tables 3 & 4 of ref. 110.

The composition may comprise one or more of these further antigens. It is preferred that combinations of antigens should be based on shared characteristics e.g. antigens associated with respiratory diseases, antigens associated with enteric diseases, antigens associated with sexually-transmitted diseases, etc.

Where a saccharide or carbohydrate antigen is used, it is preferably conjugated to a carrier protein in order to enhance immunogenicity {e.g. refs. 111 to 120}. Preferred carrier proteins are bacterial toxins or toxoids, such as diphtheria or tetanus toxoids. The CRM₁₉₇ diphtheria toxoid is particularly preferred {121}. Other carrier polypeptides include the N.meningitidis outer membrane protein {122}, synthetic peptides {123, 124}, heat shock proteins {125, 126}, pertussis proteins {127, 128}, protein D from H.influenzae {129}, cytokines {130}, lymphokines {130}, hormones {130}, growth factors {130}, toxin A or B from C.difficile {131}, iron-uptake proteins {132}, etc. Where a mixture comprises capsular saccharides from both serogroups A and C, it may be preferred that the ratio (w/w) of MenA saccharide:MenC saccharide is greater than 1 (e.g. 2:1, 3:1, 4: 1, 5:1, 10:1 or higher). Different saccharides can be conjugated to the same or different type of carrier protein. Any suitable conjugation reaction can be used, with any suitable linker where necessary.

Toxic protein antigens may be detoxified where necessary e.g. detoxification of pertussis toxin by chemical and/or genetic means {63}.

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens in the composition will typically be present at a concentration of at least 1 μg/ml each. In general, the concentration of any given antigen will be sufficient to elicit an immune response against that antigen.

As an alternative to using protein antigens in the composition of the invention, nucleic acid encoding the antigen may be used {e.g. refs. 133 to 141}. Protein components of the compositions of the invention may thus be replaced by nucleic acid (preferably DNA e.g. in the form of a plasmid) that encodes the protein.

Processes

The invention also provides a process for producing a polypeptide of the invention, comprising the step of culturing a host cell transformed with nucleic acid of the invention under conditions which induce polypeptide expression.

The invention provides a process for producing a polypeptide of the invention, comprising the step of synthesising at least part of the polypeptide by chemical means.

The invention provides a process for producing nucleic acid of the invention, comprising the step of amplifying nucleic acid using a primer-based amplification method (e.g. PCR).

The invention provides a process for producing nucleic acid of the invention, comprising the step of synthesising at least part of the nucleic acid by chemical means.

The invention also provides a process for detecting the presence of a bacterium in a sample, comprising the step of contacting the sample with nucleic acid of the invention under hybridizing conditions; and (b) detecting the presence or absence of hybridization of nucleic acid of the invention to nucleic acid present in the sample. The presence of hybridization in step (b) indicates that the sample contains the relevant bacterium.

The invention also provides an immunoassay method for detecting the presence of a bacterium, comprising the step of contacting a sample with a polypeptide or antibody of the invention.

Adhesion Inhibition

The invention provides methods for inhibiting the attachment of bacterial cells to host cells (e.g. human cells). The cell may be in vitro (e.g. in cell culture) or in vivo. The cells are most preferably human cells. The host cells will typically be epithelial or endothelial cells.

The invention provides a method for preventing the attachment of a bacterial cell to a host cell, wherein the ability of one or more of the polypeptides of the invention to bind to the host cell is blocked.

The ability to bind may be blocked in various ways but, most conveniently, an antibody specific for a polypeptide of the invention is used. As an alternative to using antibodies, antagonists of the interaction between the polypeptide of the invention and its receptor on the host cell may be used. As a further alternative, a soluble form of the host cell receptor may be used as a decoy. These can be produced by removing the receptor's transmembrane and, optionally, cytoplasmic regions.

The antibodies, antagonists and soluble receptors of the invention may be used as medicaments to prevent the attachment of a bacterial cell to a host cell.

The invention provides a method for preventing the attachment of a bacterial cell to a host cell, wherein expression of a polypeptide of the invention is inhibited. The inhibition may be at the level of transcription and/or translation. A preferred technique for inhibiting expression of the gene is antisense {e.g. refs. 142 to 148, etc.}. Antibacterial antisense techniques are disclosed in, for example, references 149 & 150.

The invention provides a method for preventing the attachment of a bacterial (e.g. Neisserial) cell to an epithelial cell, wherein the gene encoding the polypeptide of the invention is knocked out. Thus the invention provides a bacterium in which such genes have been knocked out. Techniques for producing knockout bacteria are well known. The knockout mutation may be situated in the coding region of the gene or may lie within its transcriptional control regions (e.g. within its promoter). The knockout mutation will reduce the level of mRNA encoding a polypeptide of the invention to <1% of that produced by the wild-type bacterium e.g. <0.5%, <0.1%, 0%. The knockout mutants of the invention may be used as immunogenic compositions (e.g. as vaccines). Such a vaccine may include the mutant as a live attenuated bacterium.

The invention also provides methods for screening compounds to identify those (antagonists) which inhibit the binding of a bacterial cell to a host cell.

Potential antagonists for screening include small organic molecules, peptides, peptoids, polypeptides, lipids, metals, nucleotides, nucleosides, polyamines, antibodies, and derivatives thereof. Small organic molecules have a molecular weight between 50 and about 2,500 daltons, and most preferably in the range 200-800 daltons. Complex mixtures of substances, such as extracts containing natural products, compound libraries or the products of mixed combinatorial syntheses also contain potential antagonists.

Typically, a polypeptide of the invention is incubated with a host cell and a test compound (e.g. an antibody), and the mixture is then tested to see if the interaction between the protein and the epithelial cell has been inhibited. The protein, cell and compound may be mixed in any order.

Inhibition will, of course, be determined relative to a standard (e.g. the native protein/cell interaction). Preferably, the standard is a control value measured in the absence of the test compound. It will be appreciated that the standard may have been determined before performing the method, or may be determined during or after the method has been performed. It may also be an absolute standard.

For preferred high-throughput screening methods, all the biochemical steps for this assay are performed in a single solution in, for instance, a test tube or microtitre plate, and the test compounds are analysed initially at a single compound concentration. For the purposes of high throughput screening, the experimental conditions are adjusted to achieve a proportion of test compounds identified as “positive” compounds from amongst the total compounds screened.

The method may also simply involve incubating one or more test compound(s) with a polypeptide of the invention and determining if they interact. Compounds that interact with the protein can then be tested for their ability to block an interaction between the protein and an epithelial cell.

Other methods which may be used include, for example, reverse two hybrid screening {151} in which the inhibition of the bacteria:host receptor interaction is reported as a failure to activate transcription.

The invention also provides a compound identified using these methods. These can be used to treat or prevent bacterial infection. The compound preferably has an affinity for a polypeptide of the invention of at least 10⁻⁷ M e.g. 10⁻⁸ M, 10⁻⁹ M, 10⁻¹⁰ M or tighter.

Definitions

The term “comprising” encompasses “including” as well as “consisting” e.g. a composition “comprising” X may consist exclusively of X or may include something additional e.g. X+Y.

The term “about” in relation to a numerical value x means, for example, x±10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of reference 152. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in reference 153.

### BRIEF DESCRIPTION OF THE DRAWINGS

FIGS. 1 to 15 show analyses of amino acid sequences of the invention to show coiled-coil regions.

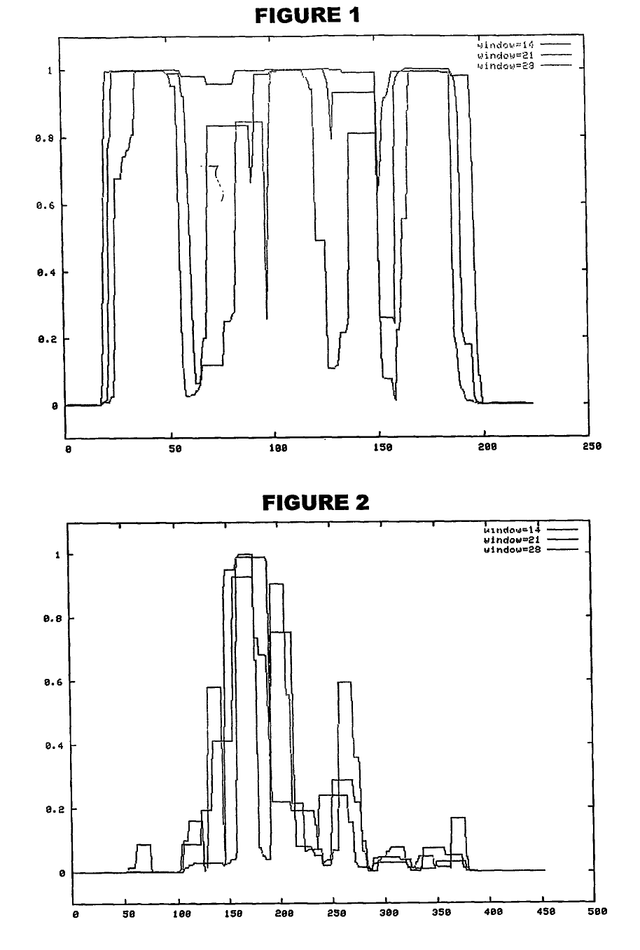

FIG. 16 shows conservation between anchor regions of polypeptides of the invention.

FIG. 17 is an illustration of the NadA structure within the meningococcal outer membrane, in monomeric and trimeric form.

FIGS. 18 & 19 show comparisons of the genetic environment of genes encoding polypeptides of the invention.

FIG. 20 illustrates the genetic environment in E.coli K1 vs. K12.

FIG. 21 shows coil analysis for (21A) NadA and (21B) HadA.

FIG. 22 is a schematic organization of the hadA locus in a hadA positive strain (F3031) and in diverse hadA negative strains (type d, type b, and several non-typeable H. influenzae).

FIG. 23 is a tree showing the relationship between HadA of different strains.

FIG. 24 illustrates three constructs for expression of HadA in E.coli.

FIG. 25 shows Bis-Tris gels of expressed HadA-His. Lanes are paired as (odd) total protein and (even) soluble proteins. Lanes 1/2 are empty plasmid at 20° C.; 3/4 are expression at 20° C.; 5/6 are empty plasmid at 30° C.; 7/8 are expression at 30° C.; 9/10 are empty plasmid at 37° C.; 11/12 are expression at 37° C.; M is pre-stained protein standard (See Blue™Plus2, Invitrogen).

FIG. 26 is a western blot. Lanes are: (1) Pre-stained protein standard, See Blue™Plus2; (2) empty plasmid, total protein, 30° C.; (3) empty plasmid, soluble protein, 30° C.; (4) expressed total protein, 30° C.; (5) expressed soluble protein, 30° C.; (6) rHad A-His.

FIG. 27 shows FACS analysis of binding to Chang cells by E.coli-expressed HadA. HadA was tested at nine concentrations and binding was assessed. Four representative FACS spectra are shown.

FIG. 28 shows phase contrast micrographs of three different aggregates in panels A to C, and cells containing empty pET plasmid in panel D.

FIG. 29 shows (A) adhesin and (B) invasion of Chang cells by E.coli expressing HadA. The left bar is control cells transformed with empty plasmid; the right bar is the HadA-expressing bacteria. Results are the mean±standard error of the mean of measurements made in triplicate.

FIG. 30 shows immunofluorescence microscopy analysis of with E.coli-pET HadA na and Chang epithelial cells. Extracellular bacteria are seen in green; intracellular bacteria are red.

FIG. 31 shows SDS-PAGE of HadA/na and HadA/LNadA/na expressed in E.coli. Lanes are: (1) Pre-stained protein standard, See Blue™Plus2; (2) empty plasmid, overnight uninduced culture; (3) HadA/na, overnight culture; (4) HadA/LNadA/na, overnight culture; (5)-(7) as for (2) to (4), but 3 hours after induction of protein expression by IPTG. Arrows show monomer and oligomer.

FIG. 32 shows a western blot of HadA/na and HadA/LNadA/na expressed in E.coli. Lanes are the same as in FIG. 31. Arrows show monomer and oligomer.

FIG. 33 shows SDS-PAGE of HadA expressed overnight in E.coli without induction. Lanes are: (1) Pre-stained protein standard, See Blue™Plus2; (2) empty plasmid; (3) HadA/na-transformed; (4) empty plasmid, outer membrane extract; (5) HadA/na-transformed, outer membrane extract.

FIG. 34 shows FACS analysis of HadA expression, showing E.coli transformed with an empty pET plasmid or with the HadA/na pET plasmid.

FIG. 35 shows the results of a settling assay in E.coli, with or without HadA expression.

### MODES FOR CARRYING OUT THE INVENTION

Neisseria meningitidis NadA Protein

Within the Neisseria meningitidis serogroup B genome {75}, an outer membrane protein (NadA) was identified {1} which shows weak homology to Yersinia enterocolitica adhesin YadA and to Moraxella catarrhalis surface protein UspA2 {154}. The nadA gene is present in a subgroup of hypervirulent N.meningitidis strains and is characterized by a low GC content, which suggests a probable acquisition event of the gene by horizontal transfer.

To investigate the possibility that proteins similar to the NadA adhesin could have been acquired by other pathogens, we searched for homologous proteins.

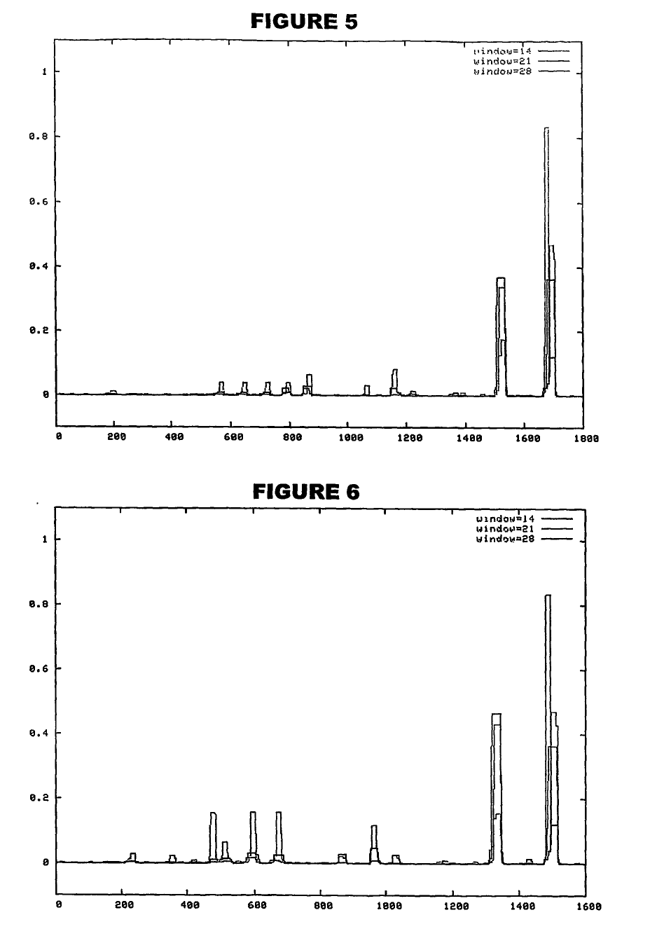

A sequence alignment of NadA & YadA revealed that the two proteins are most similar at the C-terminus, which is the membrane anchor domain. In NadA, this domain is approximately 70 residues long and contains five predicted amphipatic beta strands, which cross the outer membrane multiple times thus anchoring the protein to the surface of the bacterium (FIG. 17). Within this region, the level of sequence similarity between NadA & YadA is around 60% identity while in the N-terminal and central domain the homology is below 25% identity.

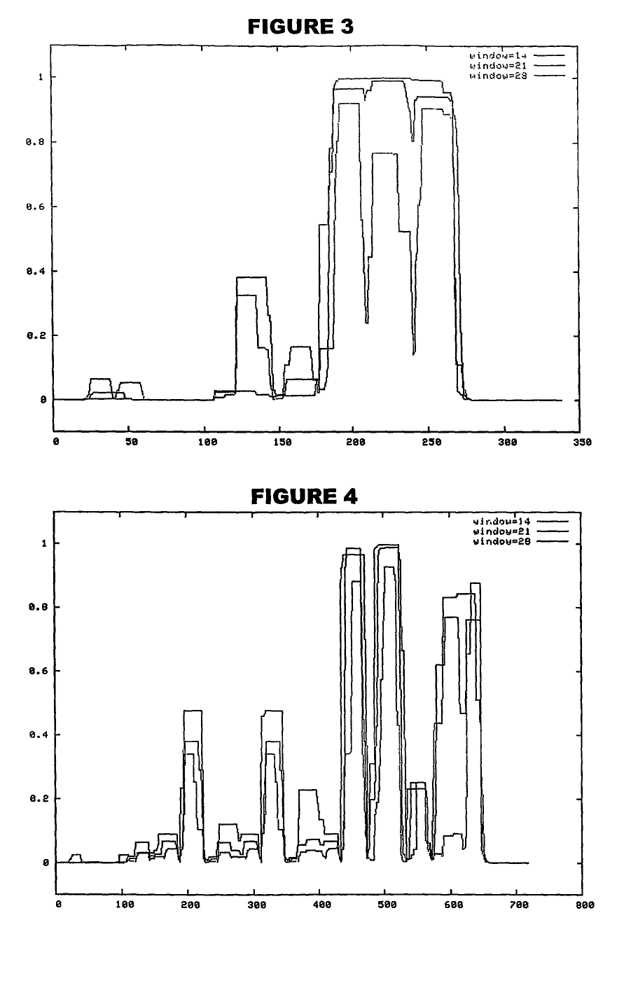

In a first search, based on the NadA anchor domain, results included YadA and UspA2, but also other proteins, such as the serum resistance protein DsrA of Haemophilus ducreyi, the immunoglobulin binding proteins EibA-C-D-E and F of E.coli, and the outer membrane protein 100 of Actinobacillus actinomycetemcomitans {154}. In order to highlight more distant members of this family, these results were used for further searches, and this approach identified 16 further results. These 16 polypeptides were further evaluated for secondary structure analysis, coiled coil prediction and presence/absence of a leader peptide. As expected, despite the little amino acid similarity displayed within the central regions, most of the identified polypeptides possess the coiled coil feature, which gives them the capability to form stable oligomers. The anchor regions of the identified polypeptides are well conserved (FIG. 16). In addition, the GC content of the genes encoding these polypeptides was lower than average for their respective genomes, suggesting that they are encoded by genes carried on mobile genetic elements.

Escherichia coli

Polypeptides were found in pathogenic strains of E.coli, including enteropathogenic (EPEC), enteroaggregative (EAEC), enterohemorragic (EHEC) and uropathogenic (UPEC) strains. Furthermore, a polypeptide almost identical to those of the EHEC and EPEC strains was found in the K1 strain, which is a capsulated E.coli strain responsible for neonatal meningitis. The K1 sequence aligns with NadA as follows:

| | |
|---|---|
| | 100 110 120 130 140 150 |
| k1.pep | TGVVQIPARYQSMINARQSAVTDAQQTQITEQQAQIVATQKTLAATGDTQNTAHYQEMIN |
| | \| :::\|\| :: \| \|::\|\| : :: \| :\| |
| NadA.pep | DAALADTDAALDETTNALNKLGENITTFAEETKTNIVKIDEKLEAVADTVD--KHAEAFN |
| | 130 140 150 160 170 180 |
| | 160 170 180 190 200 210 |
| k1.pep | ARLAAQNEANQRTTTEQGQKMNALTTDVAAQQQKERAQYDKQMQSLAQKSVQAHEQIESL |
| | : :\|:\| \| :::: \| : ::\| : ::: \| :: \| \|: \| :: |
| NadA.pep | DIADSLDETN--TKADEAVKTANEAKQTAEETKQNVDAKVKAAETAAGKAEAAAGTANTA |
| | 190 200 210 220 230 240 |
| | 220 230 240 250 260 270 |
| k1.pep | RQDSAQTQQQLTNTQKRVADNSQQINTLNNHFDSLKNEVEDNRKEANAGTASAIAIASQP |
| | : : : ::\|: : :\| \|: :\| : ::\|\|\| ::\| : \|\|\|: \| \| \|::: |
| NadA.pep | ADKAEAVAAKVTDIKADIATNKADIAKNSARIDSLDKNVANLRKETRQGLAEQAALSGLF |
| | 250 260 270 280 290 300 |
| | 280 290 300 310 320 330 |
| k1.pep | QVKTGDVMMVSAGAGTFNGESAVSVGTSFNAGTHTVLKAGISADTQSDFGAGVGVGYSF |
| | \| : : \|:\|::\| :::\|\|\|\|::\|\|:\| : : \|\|\|::: \|:\| :\|: \|\| :: |
| NadA.pep | QPYNVGRFNVTAAVGGYKSESAVAIGTGFRFTENFAAKAGVAVGTSSGSSAAYHVGVNYEW |
| | 310 320 330 340 350 360 |

24.4% identity in 209 aa overlap

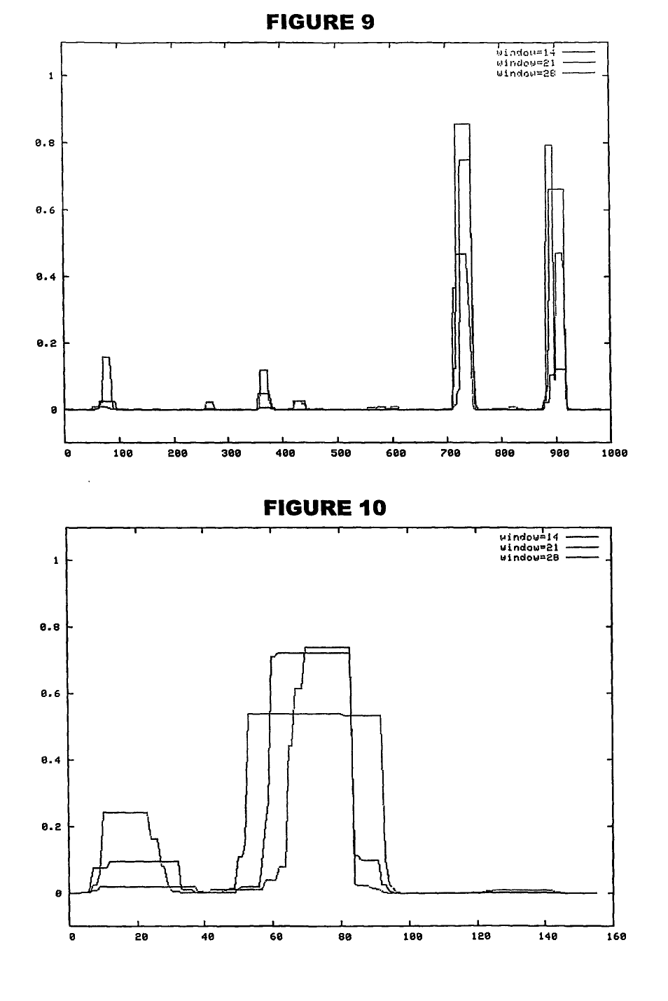

Another NadA analogue was encoded by the large virulence plasmid present in shiga toxigenic strains of E.coli (STEC) {155}. This protein (Saa) is expressed on the outer membrane of E.coli and forms high molecular weight oligomers. In contrast, no counterpart of NadA could be detected in the benign E.coli strain K12, supporting the view that these genes have been acquired by lateral exchange early during evolution of the species (FIG. 20). Nor could a counterpart be seen in laboratory strain MG1655.

Prompted by these observations, and in order to assess a possible mechanism of insertion/deletion of these genes, the arrangement of the region that harbours the gene coding for the NadA-like molecule was investigated. The sequence of this region for the EHEC strain is SEQ ID NO: 23

This analysis showed that the gene organisation of the DNA segments is almost identical among the genomes of K1, EHEC and EPEC, with a sequence conservation of the NadA-like proteins that ranges from 95% identity between K1 and EHEC to 98% identity between K1 and EPEC. In the case of EAEC, although the flanking regions are conserved, the sequence of the NadA-like protein is 380 residues longer than the others, even if the N-terminus and C-terminus are well conserved.

| Bacterium | Amino acid | Nucleic acid | Figure |
|---|---|---|---|
| E. coli K1 & E. coli EHEC | SEQ ID NO: 2 | SEQ ID NO: 22 | 3 |
| strain EDL933 | | | |
| E. coli EPEC strain | SEQ ID NO: 7 | SEQ ID NO: 24 | — |
| E2348/69 | | | |
| E. coli EAEC strain O42 | SEQ ID NO: 8 | SEQ ID NO: 25 | 4 |

Extending the analysis to the K12 genome, the insertion site was found to be between two hypothetical open reading frames (YbbJ and YbbI) coded on opposite strands, and that the small “island” consists of three genes: an ORF coding for an hypothetical integral membrane protein, the gene for the putative NadA-like adhesin, and an ORF for a predicted lipoprotein of unknown function. The two latter ORFs are probably co-transcribed, while the first one is coded on the reverse orientation. A couple of 7-bp direct repeats (CTGACGC) that could represent putative insertion sites could be mapped at the boundaries of the inserted fragments (SEQ ID NO: 23, starting at nucleotides 1811 & 4255), and this repeat is absent in the vicinities of the point of insertion in the K12 strain.

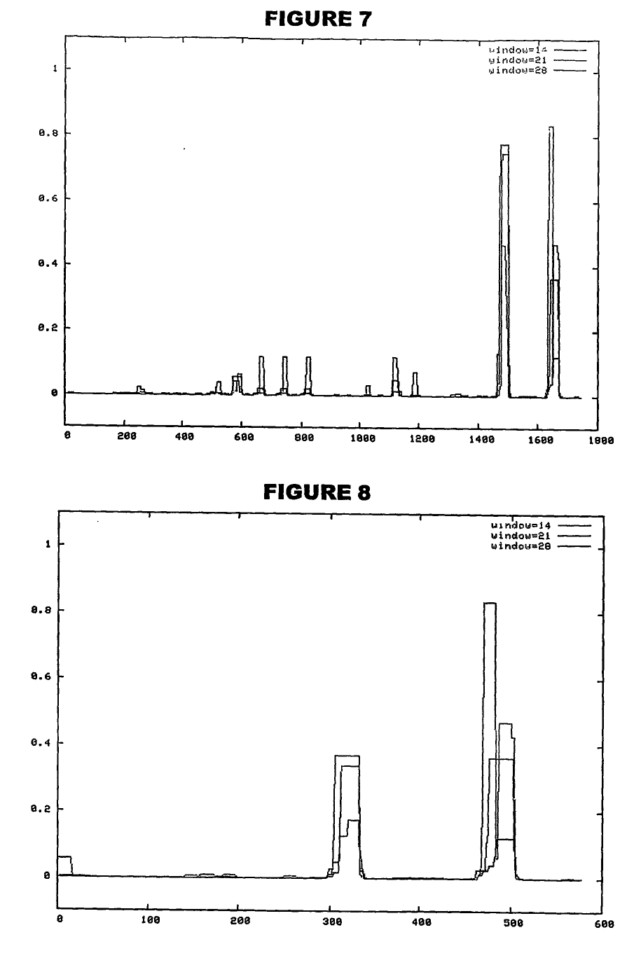

The length of the acquired DNA regions is 2348 bases for EPEC, 2450 bases for K1 and EHEC, and 2630 for EAEC (FIG. 18). In all cases, the G+C content of the fragment is lower if compared to the average composition calculated for each genome, thus confirming the preliminary hypothesis that this segment has been acquired by pathogenic E.coli by a mechanism of lateral transfer.

In the case of uropathogenic E.coli (UPEC), a different DNA segment was found between the ybbJ ad ybbI genes. This segment is 1342 bp long and encodes a predicted cytoplasmic protein, which is conserved only in Salmonella typhymurium LT2, but absent from all the other analyzed strains of E.coli. Differently from the other described insertion fragments, no direct repeats could be mapped at the boundaries of this island, whose GC composition is also very similar to the average value. These data could indicate that the NadA-like encoding gene has been inserted later on in place of the c0608 gene. Nevertheless, subsequent search revealed that a gene coding for an homologue of NadA could be found in a different location of the genome of uropathogenic E.coli strain CFT073. This protein is more distantly related to NadA and is seen as a member of a second NadA-like family of proteins. Counterparts of this protein are contained in the other pathogenic strains of E.coli at analogous locations and, similarly to the first group of E.coli NadA-like molecules, the corresponding genes are also encoded on small islands and are not present in the K12 strain (FIG. 19). Furthermore, these genes have strong similarities at the 3′ end with a frame-shifted Shigella flexneri sequence. The arrangement of NLM flanking regions has been compared in the two species (E.coli and Shigella) revealing striking similarities. Although the sequence conservation is restricted to the amino and carboxy-terminal portions of the adhesin coding genes, the flanking regions are syntenic and share more than 80% identity at the nucleotide level. Upstream of the NadA-like gene, this island contains an ORF coding for a lipoprotein that is frameshifted either in EPEC, EHEC and in Shigella. Furthermore, in the genome of Shigella, two additional genes (insA and insB), coding for transposase elements are found in the vicinities of the NLM gene.

| Bacterium | Amino acid | Nucleic acid | Figure |
|---|---|---|---|
| E. coli UPEC strain | SEQ ID NO: 10 | SEQ ID NO: 26 | 5 |
| CFT073 | | | |
| E. coli EHEC | SEQ ID NO: 3 | SEQ ID NO: 27 | 6 |
| E. coli EAEC | SEQ ID NO: 9 | SEQ ID NO: 28 | 7 |
| E. coli EPEC | SEQ ID NO: 18 | SEQ ID NO: 30 | 8 |
| S. flexneri | SEQ ID NO: 11 | SEQ ID NO: 31 | 9 |

Haemophilius

An incomplete NadA homolog was found in Brazilian purpuric fever (BPF) Haemophilus influenzae isolates {156}. This polypeptide has been named HadA. NadA and HadA align as follows:

| | |
|---|---|
| | 10 20 30 40 |
| HadA pep | MKRNLLKQSVIAVLIGGTTVSNYALAQAQAQAQVKKDELSELKKQVKEM- |
| | :: \|\|\|:::\| : : \|::\| ::: : |
| NadA.pep | KTVNENKQNVDAKVKAAESEIEKLTTKLADTDAALADTDAALDETTNALNKLGENITTFA |
| | 100 110 120 130 140 150 |
| | 50 60 70 80 90 100 |
| HadA.pep | DAAIDGILDDNIAYEAEVDAKLDQHSAALGRHTNRLNNLKTIAEKAKGDSSEALDKIEAL |
| | : : :\|: : \|\| :\|: :\|:\|: \|:: :: \|:: :\| \|::\| ::\|\| : \| |
| NadA.pep | EETKTNIVKIDEKLEAVADT-VDKHAEAFNDIADSLDETNTKADEAVKTANEAKQTAEET |
| | 160 170 180 190 200 210 |
| | 110 120 130 140 150 160 |
| HadA.pep | EEQNDEFLADITALEEGVDGLDDDIAGIQDNISD----IEDDINQNSADIATNTAAIATH |
| | ::: \| \| : \| \| :: \| : \|\| :: :\| : ::: :\|\|\|\|\|\| \| \|\| : |
| NadA.pep | KQNVD---AKVKAAETAA-GKAEAAAGTANTAADKAEAVAAKVTDIKADIATNKADIAKN |
| | 220 230 240 250 260 270 |
| | 170 180 190 200 210 220 |
| HadA.pep | TQRLDNLDNRVNNLNKDLKRGLAAQAALNGLFQPYNVGKLNLTAAVGGYKSQTAVAVG |
| | : \|:\|:\|\|: \| \|\| \|: ::\|\|\| \|\|\|\|:\|\|\|\|\|\|\|\|\|::\|:\|\|\|\|\|\|\|\|\|::\|\|\|:\| |
| NadA.pep | SARIDSLDKNVANLRKETRQGLAEQAALSGLFQPYNVGRFNVTAAVGGYKSESAVAIGTG |
| | 280 290 300 310 320 330 |
| NadA.pep | FRFTENFAAKAGVAVGTSSGSSAAYHVGVNYEW |
| | 340 350 360 |

No HadA counterpart could be detected either in non-typeable H.influenzae strain 86028, which is responsible for otitis media in children, or in the non-pathogenic H.influenzae strain Rd KW20. The very high level of sequence identity between HadA and NadA in the C-terminal anchor region might indicate a common origin.

In order to analyze the origin of the hadA gene, the nucleotide sequence of this DNA region in the BPF isolate (SEQ ID NO: 20) was compared to the same region in the genome sequence for H.influenzae strains: the non-pathogenic strain Rd {157}, and a non-typeable 86028 strain (NTHi 86028), associated with pediatric otitis media disease.

The results of this comparison indicate that the adhesin coding gene is specific for the Brazilian Purpuric Fever clone (strain F3031), while no counterparts could be mapped either in the laboratory Rd or in the non-typeable strains. The HadA-encoding fragment has an organization that closely resembles that described for NadA {1} and includes an intact open reading frame plus a 182 bp upstream region, which contains −10 and −35 promoter elements. The small genetic island is flanked by the RNA helicase gene at the 5′ end and by a putative protease encoding gene located at the 3′ end. The GC composition of the recombined segment is consistent with the rest of the genome.

In contrast, while the NTHi 86028 strain can be regarded as a totally negative strain as it lacks the whole region encompassing the RNA helicase and protease ORFs, the Rd genome contains at this location a DNA segment of 1.1 kb, which encodes two short ORFs of unknown function. This region is characterized by an abnormal GC content (32%) thus suggesting that an independent ace at this site.

Additional NadA-like molecules were identified in other Haemophilus species, namely H.somnus, H.ducreyi and H.actinomycetemcomitans (also known as Actinobacillus actinomycetemcomitans).

| Bacterium | Amino acid | Nucleic acid | Figure |
|---|---|---|---|
| H. influenzae biogroup | SEQ ID NO: 1 | SEQ ID NO: 20 | 1 |
| aegyptius | | | |
| H. somnus strain 129PT | SEQ ID NO: 5 | SEQ ID NO: 21 | 2 |
| H. ducreyi | SEQ ID NO: 6 | — | — |
| H. actinomycetemcomitans | SEQ ID NO: 4 | — | — |

NadA and the H.actinomycetemcomitans sequence align as follows:

| | |
|---|---|
| | 10 20 30 40 50 |
| actac.pe | MTYQLFKHHLVALMVTGAISVNALAKDSFLENPSANLPQQVFKNR--VD--IFNNETNI |
| | \|:: :\|\| : \| :\|: \|\| : \|::\| |
| NadA.pep | TIYDIGEDGTITQKDATAADVEADDFKGLGLKKVVTNLTKTVNENKQNVDAKVKAAESEI |
| | 60 70 80 90 100 110 |
| | 60 70 80 90 100 110 |
| actac.pe | NENKKDIAINKANIASIEKDVMRNTGGIDRLAKQELVNRARITKNELDIRKNTKSIAENT |
| | :: :\| : \| :\|: : : ::\|:::::\|::: \|\| : : \| :\| \|: |
| NadA.pep | EKLTTKLADTDAALADTDAALDETTNALNKLGEN-------ITTFAEETKTNIVKIDEKL |
| | 120 130 140 150 160 170 |
| | 120 130 140 150 160 |
| actac.pe | ASIA-RIDGNLEGVNRVLQNVDVRSTE--------NAARSRANE--QKIAENKKAIENKA |
| | ::\| :\| : \|: \| : :::\| :\|: \| \|:: \|:\| \|:: : \|\| \|: \| |
| NadA.pep | EAVADTVDKHAEAFNDIADSLDETNTKADEAVKTANEAKQTAEETKQNVDAKVKAAETAA |
| | 180 190 200 210 220 230 |
| | 170 180 190 200 210 220 |
| actac.pe | DKADVEKNRADIAAN-SRAIAT-FRSSSQNIAALTTKVDRNTARIDRLDSRVNELDKEVK |
| | \|\|:: : \|: \|\|: ::\|:\|: : : :\|\|: : : :\|:\|\|\|\| \|\|: \| :\| \|\|:: |
| NadA.pep | GKAEAAAGTANTAADKAEAVAAKVTDIKADIATNKADIAKNSARIDSLDKNVANLRKETR |
| | 240 250 260 270 280 290 |
| | 230 240 250 260 270 280 |
| actac.pe | NGLASQAALSGLFQPYNVGSLNLSAAVGGYKSKTALAVGSGYRFNQNVAAKAGVAVSTN- |
| | :\|\|\| \|\|\|\|\|\|\|\|\|\|\|\|\|\| :\|::\|\|\|\|\|\|\|\|::\|:\|:\|:\|:\|\|::\| \|\|\|\|\|\|\|\|:\|: |
| NadA.pep | QGLAEQAALSGLFQPYNVGRFNVTAAVGGYKSESAVAIGTGFRFTENFAAKAGVAVGTSS |
| | 300 310 320 330 340 350 |
| | 290 |
| actac.pe | GGSATYNVGLNFEW |
| | \|:\|\|:\|:\|\|:\|:\|\| |
| NadA.pep | GSSAAYHVGVNYEW |
| | 360 |

37.0% identity in 284 aa overlap

NadA and the H.somnus sequence align as follows:

| | |
|---|---|
| | 90 100 110 120 130 140 |
| H.somnus.pep | EVIKGWNEVKSLPRIDGNGKDKQTKDQIAMLIRTVDNTKELGRIVSTNIEDIKNLKKELY |
| | \| \| \|: :: : ::: \| \| :\|: |
| NadA.pep | MSMKHFPSKVLTTAILATFCSGALAATSDD--DVKKAATVAIVAAYNNGQEIN |
| | 10 20 30 40 50 |
| | 150 160 170 180 190 |
| H.somnus.pep | GF-----VEDVNES---EARNISRIDENEKDIKNL--KKELYDFVEDVNESEARNISRID |
| | \|\| : \|::\|: :: : \| : \|:\|:\| \|\| : :::: \|\|\|:: ::: |
| NadA.pep | GFKAGETIYDIGEDGTITQKDATAADVEADDFKGLGLKKVVTNLTKTVNENKQNVDAKVK |
| | 60 70 80 90 100 110 |
| | 200 210 220 230 240 250 |
| H.somnus.pep | ENEKDINTLK-ELMDED--LNSVLTQIEDVKLTFQDVNDNVNLAFEEINGNAQKFDTAIE |
| | \|::\|: \| :\| \| \| \| :: : :::: ::: :::\|:: \|\| : \| \|:\| :\| |
| NadA.pep | AAESEIEKLTTKLADTDAALADTDAALDETTNALNKLGENITTFAEETKTNIVKIDEKLE |
| | 120 130 140 150 160 170 |
| | 260 270 280 290 300 310 |
| H.somnus.pep | GLTSGLSDLQAKVDANKQETEDDIADNAKAIHSNTKGIAKNTKDIRDLDTKTKQMLENDK |
| | :::: \|\| : \|: :\|\|\|\|: :::: :\|:::: :: :\|\|\| |
| NadA.pep | AVAD-------TVDKHA-EAFNDIADSLDETNTKADEAVKTANEAKQTAEETKQ------ |
| | 180 190 200 210 |
| | 320 330 340 350 360 370 |
| H.somnus.pep | NLMTGLESLATETSKGFERFDVKTQQLDQAVANVVGRVDITEQAIRQNTAGLVNVNKRVD |
| | \|: : ::: \| ::\|: : : \|:\| \| \|:::\| : \| \| \| ::: : \|:\| |
| NadA.pep | NVDAKVKAAETAAGKAEAAAGTANTAADKAEA-VAAKVTDIKADIATNKADIAKNSARID |
| | 220 230 240 250 260 270 |
| | 380 390 400 410 420 |
| H.somnus.pep | TLDKN-------TKAGIASAVALGMLPQSTAPGKSLVSLGVGHHRGQSATAIGVSSMSSN |
| | :\|\|\|\| \|: \|:\| :\|\|: \| \| \|: \|: :\|\| ::::\|\|:\|\|\| ::: : |
| NadA.pep | SLDKNVANLRKETRQGLAEQAALSGLFQPYNVGRFNVTAAVGGYKSESAVAIG-TGFRFT |
| | 280 290 300 310 320 330 |
| | 430 440 450 |
| H.somnus.pep | GKWVVKGGMSYDTQRHATFGGSVGFFFN |
| | ::::\|:\|:: \|: :: : \|\| |
| NadA.pep | ENFAAKAGVAVGTSSGSSAAYHVGVNYEW |
| | 340 350 360 |

23.2% identity in 354 aa overlap

NadA and the H.ducreyi sequence align as follows:

| | |
|---|---|
| | 150 160 170 180 190 200 |
| H.ducreyi.pe | SKNKQNIDTISKYLLELGTYLDGSYRMMEQNTHNINKNTHNINKNTHNINKLSKELQTGL |
| | \| :\| \|\|: \|:: :\|: :\| \|\| : \|\| |
| NadA.pep | EAAAGTANTAADKAEAVAAKVTDIKADIATNKADIAKNSARIDSLDKNVANLRKETRQGL |
| | 240 250 260 270 280 290 |
| | 210 220 230 240 250 260 |
| H.ducreyi.pe | ANQSALSMLVQPNGVGKTSVSAAVGGYRDKTALAIGVGSRITDRFTAKAGVAFNTYNGG- |
| | \|:\|:\|\|\| \| \|\| :\|\|: :\|:\|\|\|\|\|\|::::\|:\|\|\|:\| \|:\|: \|:\|\|\|\|\|\| :\| :\|: |
| NadA.pep | AEQAALSGLFQPYNVGRFNVTAAVGGYKSESAVAIGTGFRFTENFAAKAGVAVGTSSGSS |
| | 300 310 320 330 340 350 |
| | 270 |
| H.ducreyi.pe | MSYGASVGYEF |
| | :\| ::\|:\|\|: |
| NadA.pep | AAYHVGVNYEW |
| | 360 |

47.5% identity in 101 aa overlap

NB: the coiled prediction for the H.ducreyi polypeptide is not high.

Other Bacteria

Further NadA homologs identified in the search are:

| Bacterium | Amino acid | Nucleic acid | Figure |
|---|---|---|---|
| Brucella melitensis | SEQ ID NO: 12 | SEQ ID NO: 32 | 10 |
| Brucella suis | SEQ ID NO: 13 | SEQ ID NO: 33 | 11 |
| Ralstonia solanacearum | SEQ ID NO: 14 | SEQ ID NO: 34 | 12 |
| Sinorhizobium meliloti | SEQ ID NO: 15 | SEQ ID NO: 35 | 13 |
| Bradorhizobium | SEQ ID NO: 16 | SEQ ID NO: 36 | 14 |
| japonicum | | | |
| Burkholderia fungorum | SEQ ID NO: 17 | SEQ ID NO: 29 | 15 |

Multiple Sequence Alignment

A multiple sequence alignment of members of the NadA “family” is below:

| | | |
|---|---|---|
| | 10 20 30 40 50 60 | |
| | \| \| \| \| \| \| | |
| 961_HI | ------MKRNLLKQSVIAVLIGGTTVSN-------------------------------- | SEQ ID NO: 1 |
| 961_ACTAC | ------MTYQLFKHHLVALMVTGAISVNAL------------------------------ | SEQ ID NO: 4 |
| MenB NadA | -MSMKHFPSKVLTTAILATFCSGALAATSDDDVKK---AATVAIVAAYNNGQEINGFKAG | SEQ ID NO: 37 |
| YADA_YEREN | --MTKDFKISVSAALISALFSSPYAFADDYDGIPN---LTAVQISPNADPALGLEYPVRP | SEQ ID NO: 38 |
| 961_HAESO | MKKVQFFKYSSLALALGLGVSASALAAPTSTSTTTGPEAPPTGPAPTAKDPLAETALAYD | SEQ ID NO: 5 |
| 961_K1 | ---MKTVNVALLALIISATSSPVVLAGDTIEAAAT------------------------- | SEQ ID NO: 2 |
| 961_HAEDU | ------MKIKCLVAVVGLACSTITTMAQQP------------------------------ | SEQ ID NO: 6 |
| | . : | |
| Prim.cons. | M23MK42K22LLA2AI2A2FS2GALAA2T6D444TGPEA33V3I3P3A333L33333333 | SEQ ID NO: 39 |
| | 70 80 90 100 110 120 | |
| | \| \| \| \| \| \| | |
| 961_HI | YALAQAQAQAQVKKD | |
| 961_ACTAC | AKDSFLENPSANLPQQVFKNR---VDIFNNET------------------- | |
| MenB NadA | ETIYDIGEDGTITQKDATAADVEADDFKGLGLKKVVTNLTK------------------- | |
| YADA_YEREN | PVPGAGGLNASAKGIHSIAIGATAEAAKGAAVAVGAGSIATGVNSV-----------AIG | |
| 961_HAESO | LENEVAYLRMKAGEWMQLGLDPEKEVIKGWNEVKSLPRIDGNGKDKQTKDQIAMLIRTVD | |
| 961_K1 | --------ELSAINSGMSQSEIEQKITRFLERTDNSPAAYT------------------- | |
| 961_HAEDU | ----------PKFAGVSSLYSYEYDYGKGK------------------------------ | |
| | . : | |
| Prim.cons. | 333333GL4A2A6677SS2ADAEA3VFKGL444255PNI5T22222QTKDQIAMLIR222 | |
| | 130 140 150 160 170 180 | |
| | \| \| \| \| \| \| | |
| 961_HI | ELSELKKQVKEMDAAIDGILDDNIAYEAEVDAKLDQHSAALGRHTNRLNNLKT------- | |
| 961_ACTAC | NINENKKDIAINKANIASIEKDVMRNTGGIDRLAKQELVNRARITKNELDIR-------- | |
| MenB NadA | TVNENKQNVDAKVKAAESEIEKLTTKLADTDAALADTDAALDETTNALNKLGE------- | |
| YADA_YEREN | PLSKALGDSAVTYGAASTAQKDGVAIGARASTSDTGVAVGFNSKADAKNSVAIGHSSHVA | |
| 961_HAESO | NTKELGRIVSTNIEDIKNLKKELYGFVEDVNESEARNISRIDENEKDIKNLKK------- | |
| 961_K1 | YLTEHHYIPSETPDTTQTPPVQTDPDAGQKTVAATGVVQIPARYQSMINARQS------- | |
| 961_HAEDU | WTWSNEGGFDIKVPGIKMKPKEWISKQATYLELQHYMPYTPVLVTSAPDVSPS------- | |
| | . . . | |
| Prim.cons. | NL2ENK22V323VAAIK2IPKDLIAK7ADVD23222V72A22R7T3A2NNLKSGHSSHVA | |
| | 190 200 210 220 230 240 | |
| | \| \| \| \| \| \| | |
| 961_HI | ----------------------------------IAEKAKGDSSEALDKIEALEEQNDE- | |
| 961_ACTAC | ----------------------------------KNTKSIAENTASIARIDGNLEGVNR- | |
| MenB NadA | ----------------------------------NITTFAEETKTNIVKIDEKLEAVADT | |
| YADA_YEREN | ANHGYSIAIGDRSKTDRENSVSIGHESLNRQLTHLAAGTKDTDAVNVAQLKKEIEKTQEN | |
| 961_HAESO | ------------------------------ELYDFVEDVNESEARNISRIDENEKDINTL | |
| 961_K1 | ---------------------------------AVTDAQQTQITEQQAQIVATQKTLAAT | |
| 961_HAEDU | -----------------------------------SISILLYPMSDPDQLGINRQQLKLN | |
| | :: : | |
| Prim.cons. | ANHGYSIAIGDRSKTDRENSVSIGHESLNR2L236A2K7KEE72ENIAQID2N2EQ22E2 | |
| | 250 260 270 280 290 300 | |
| | \| \| \| \| \| \| | |
| 961_HI | -------------FLADITALEEG----------------------VDGLDDDIAGIQDN | |
| 961_ACTAC | -------------VLQNVDVRSTENAA------------------RSRANEQKIAENKKA | |
| MenB NadA | VDKHAEAFNDIADSLDETNTKADEAVK------------------TANEAKQTAEETKQN | |
| YADA_YEREN | TNKRS------AELLANANAYADNKSSSV-LGIANNYTDSKSAETLENARKEAFAQSKDV | |
| 961_HAESO | KELMDEDLNSVLTQIEDVKLTFQDVNDNVNLAFEEINGNAQKFDTAIEGLTSGLSDLQAK | |
| 961_K1 | GDTQN---TAHYQEMINARLAAQNEAN----------------QRTTTEQGQKMNALTTD | |
| 961_HAEDU | ----------LYSYFNDLRHDFK-----------------------LKVLDARISKNKQN | |
| | : : | |
| Prim.cons. | 4DK44E22N34257LA22227A225A52VNL2222222222223TT7N3L2QKIAE2K2N | |
| | 310 320 330 340 350 360 | |
| | \| \| \| \| \| \| | |
| 961_HI | ISDIED-----------------------------------DINQNSADIATNTAAIATH | |
| 961_ACTAC | IENKADKA---------------------------------DVEKNRADIAANSRAIATF | |
| MenB NadA | VDAKVKAA---------------------------------ETAAGKAEAAAGTANTAAD | |
| YADA_YEREN | LNMAKAHSNSVARTTLETAEEHANSVAR-----TTLETAEEHANKKSAEALASANVYADS | |
| 961_HAESO | VDANKQETEDDIADNAKAIHSNTKGIAKNTKDIRDLDTKTKQMLENDKNLMTGLESLATE | |
| 961_K1 | VAAQQQKE--------------------------------RAQYDKQMQSLAQKSVQAHE | |
| 961_HAEDU | IDTISK-----------------------------------YLLELGTYLDGSYRMMEQN | |
| | : | |
| Prim.cons. | 2DA2K3KA222222222222222222A2NTKDI22L2T223D722NSA23AA3T22IATE | |
| | 370 380 390 400 410 420 | |
| | \| \| \| \| \| \| | |
| 961_HI | TQRLDNLDNRVNNLNKDLKRGLAA | |
| 961_ACTAC | RSS---SQ---------NIAALTTKVDR-------NTARIDRLDSRVNELDKEVKNGLAS | |
| MenB NadA | KAE---AVAAKVTDIKADIATNKADIAK-------NSARIDSLDKNVANLRKETRQGLAE | |
| YADA_YEREN | KSS---HTLKTANSYTDVTVSNSTKKAIRESNQ-YTDHKFRQLDNRLDHLDTRVDRGLAS | |
| 961_HAESO | TSKGFERFDVKTQQLDQAVANVVGRVDITEQAIRQNTAGLVNVNKRVDTLDKNTKAGIAS | |
| 961_K1 | QIES---LRQDSAQTQQQLTNTQRRVADNSQQINTLNNHFDSLKNEVEDNRKEANAGTAS | |
| 961_HAEDU | THN------------------IN-----------KNTHNINKNTHNINKLSKELQTGLAN | |
| | : .: . * * | |
| Prim.cons. | 2S22FE4544K44Q44Q5IANN6T2VAI3EQ3I24NTARID2LDNRVN2LDKE3KAGLAS | |
| | 430 440 450 460 470 480 | |
| | \| \| \| \| \| \| | |
| 961_HI | QAALWGLFQPYNVGKLNLTAAVGGYKSQTAVAVG-------------------------- | |
| 961_ACTAC | QAALSGLFQPYNVGSLNLSAAVGGYKSKTALAVGSG-YRFNQNVAAHAGVAVST-N-GGS | |
| MenB NadA | QAALSGLFQPYNVGRFNVTAAVGGYKSESAVAIGTG-FRFTENFAAKAGVAVGTSS-GSS | |
| YADA_YEREN | SAALNSLFQPYGVGKVNFTAGVGGYRSSQALAIGSG-YRVNHNVALKAGVAYAG---SSD | |
| 961_HAESO | AVALGMLPQSTAPGKSLVSLGVGHHRGQSATAIGVSSMSSNGKWVVKGGMSYDTQR-HAT | |
| 961_K1 | AIAIASQPQVKTGDVMMVSAGAGTFNGESAVSVGTS-FNAGTHTVLKAGISADTQS-DFG | |
| 961_HAEDU | QSALSMLVQPNGVGKTSVSAAVGGYRDKTALAIGVG-SRITDRFTAKAGVAFNTYNGGMS | |
| | *: * . .: ..* .... * ::* :::*:*::: : | |
| Prim.cons. | QAALSGLFQPYNVGKLNVSAAVGGY2S32A2AIG3GS2RFNEN2AAKAGVA2DTQ2GGSS | |
| | 490 | |
| | \| | |
| 961_HI | ----------- | |
| 961_ACTAC | ATYNVGLNFEW | |
| MenB NadA | AAYHVGVNYEW | |
| YADA_YEREN | VMYNASFNIEW | |
| 961_HAESO | --FGGSVGFFFN | |
| 961_K1 | --AGVGVGYSF | |
| 961_HAEDU | --YGASVGYEF | |
| | : :: : : | |
| Prim.cons. | AGY2VGVNFEW | |

HadA Studies

As mentioned above, the HadA sequence (SEQ ID NO: 1) was initially found in an incomplete form. The complete HadA locus was amplified using a forward oligonucleotide primer HOM F (SEQ ID NO: 40), a reverse primer HOM R2 (SEQ ID NO: 41) and an alternative reverse primer HOM R3 (SEQ ID NO: 42) that is further downstream than HOM R2. Seven primers (forward: SEQ ID NOS: 43 to 46; reverse: SEQ ID NOS: 47 to 49) were used to sequence the complete locus.

The complete locus in F3031 strain is given as SEQ ID NO: 50. Nucleotides 874 to 1339 of this sequence are new and downstream of SEQ ID NO: 20. The amino acid sequence of HadA is SEQ ID NO: 51. The C-terminus downstream of SEQ ID NO: 20 is given separately as SEQ ID NO: 52.

An alignment of NadA and HadA (39.5% identity in 243 aa overlap) is given below:

| | |
|---|---|
| | 10 20 30 40 50 60 |
| HadA | MKRNLLKQSVIAVLIGGTTVSNYALAQAQAQAQVKKDELSELKKQVKEM-DAAIDGILDDNIAYEAEVDA |
| | :: \|\|\|:::\| : : \|::\| ::: : : : :\|: : \|\| :\|: |
| NadA | FKGLGLKKVVTNLTKTVNENKQNVDAKVKAAESEIEKLTTKLADTDAALADTDAALDETTNALNKLGENITTFAEETKTNIVKIDEKLEAVADT |
| | 80 90 100 110 120 130 140 150 160 170 |
| | 70 80 90 100 110 120 130 140 150 |
| | KLDQHSAALGRHTNRLNNLKTIAEKAKGDSSEALDKIEALEEQNDEFLADITALEEGVDGLDDDITGIQDNISD----IEDDINQNSADIATNT |
| | :\|:\|: \|:: :: \|:: :\| \|::\| ::\|\| : \| ::: \| \| : \| \| :: \| : :\| :; :\| : ::: :\|\|\|\|\|\| |
| | -VDKHAEAFNDIADSLDETNTKADEAVKTANEAKQTAEETKQNVD---AKVKAAETAA-GKAEAAAGTANTAADKAEAVAAKVTDIKADIATNK |
| | 180 190 200 210 220 230 240 250 260 |
| | 160 170 180 190 200 210 220 230 240 |
| | AAIATHTQRLDNLDNRVNNLNKDLKRGLAAQAALNGLFQPYNVGKLNLTAAVGGYKSQTAVAVGTGYRYNENIAAKAGVAF--THGGSATYNVG |
| | \| \|\| :: \|:\|:\|\|: \| \|\| \|: ::\|\|\| \|\|\|\|:\|\|\|\|\|\|\|\|\|::\|:\|\|\|\|\|\|\|\|\|::\|\|\|:\|\|\|:\|::\|\|:\|\|\|\|\|\|\| : \|:\|\|:\|:\|\| |
| | ADIAKNSARIDSLDKNVANLRKETRQGLAEQAALSGLFQPYNVGRFNVTAAVGGYKSESAVAIGTGFRFTENFAAKAGVAVGTSSGSSAAYHVG |
| | 270 280 290 300 310 320 330 340 350 |
| | 250 |
| | VNFEW |
| | \|\|:\|\| |
| | VNYEW |
| | 360 |

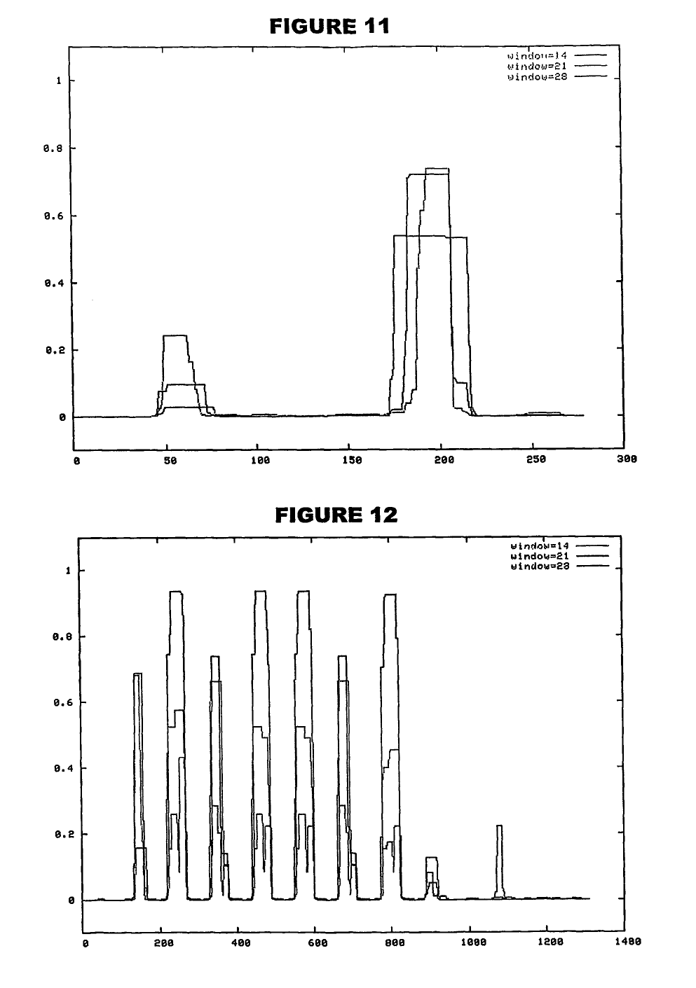

Although the overall identity is 39.5%, the identity in the C-terminus portion is much higher (up to 86%). Although the central domains of the two proteins are not well conserved, both proteins are predicted to adopt a strong coiled-coil conformation (FIGS. 21A & 21B).

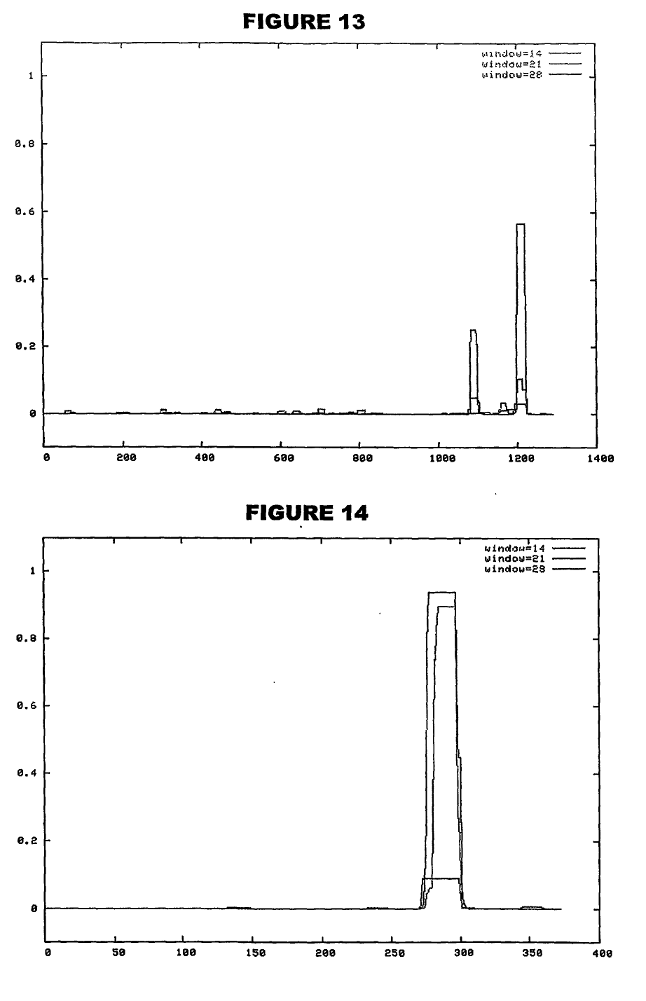

A schematic organization of the hadA locus in a hadA positive strain (F3031) and in diverse hadA negative strains (type d, type b, and several non-typeable H. influenzae) is shown in FIG. 22. The flanking genes are always conserved: they are HI0422, a RNA helicase and HI0419, a putative protease, both in a reverse orientation with respect to hadA.

Immediately downstream of hada is a gene encoding a hypothetical protein (SEQ ID NOS: 53 & 54), which is frame-shifted in strain KW20 and absent from all other Haemophilus strains tested. The closest database match for this protein is ZP_{—}00132218.1, the histone acetyltransferase HPA2 and related acetyltransferases from Haemophilus somnus 2336 (SEQ ID NO: 55):

| Length = 168 | | |
|---|---|---|
| Score = 276 bits (707), Expect = 9e−74 | | |
| Identities = 139/168 (82%), Positives = 149/168 (88%) | | |
| Query: 1 | MINENLAYLSVLPLEDVKIERSSFSCSVEPLENYFHKYVSQDVKKGLAKCFVLINAQPSR | 60 |
| | MINENL YLSVLPLED+ I+R+SFSCSVEPLE YF+KY SQDVKKG+ KCFVLIN Q | |
| Sbjct: 1 | MINENLPYLSVLPLEDLTIDRNSFSCSVEPLETYFYKYASQDVKKGITKCFVLINKQQFG | 60 |
| Query: 61 | IVGYYTLSALSIPIPDIPQERISKGVPYPNIPAVLIGRLAIDTNFQKQGYGKFLIADAIH | 120 |
| | I+GYYTLSALSIPI DIPQERISKG+PYPNIPAVL+GRLAIDTNFQ QGYGKFLIADAI+ | |
| Sbjct: 61 | IIGYYTLSALSIPITDIPQERISKGIPYPNIPAVLVGRLAIDTNFQNQGYGKFLIADAIY | 120 |
| Query: 121 | KIKNATVAATILVVEAKNDDASSFYERLGFIEFKEFGGTHRKLFYPLT | 168 |
| | KIKNATV A ILVVEAKND A SFY+RLGFIEFK THRKLFYPLT | |
| Sbjct: 121 | KIKNATVGAAILVVEAKNDHAVSFYKRLGFIEFKNLKKTHRKLFYPLT | 168 |

An alignment of the locus in HadA-negative strains is given below:

CLUSTAL W (1.83) multiple sequence alignment

| | | |
|---|---|---|
| 86028 | GCAAGCCAAGTAACAGTAATGTTTAATTAGGTATGATTTAAATTCTGTTTTATATCACAC | SEQ ID NO: 56 |
| R2846 | GCAAGCCAAGTAACAGTAATGTTTAATTAGGTATGATTTAAATTCTGTTTTATATCACAC | SEQ ID NO: 57 |
| NT36 | GCAAGCCAAGTAACAGTAATGTTTAATTAGGTATGATTTAAATTCTGTTTTATATCACAC | SEQ ID NO: 58 |
| EAGAN | GCAAGCCAAGTAACAGTAATGTTTAATTAGGTATGATTTAAATTCTGTTTTATATCACAC | SEQ ID NO: 59 |
| HK707 | GCAAGCCAAGTAACAGTAATGTTTAATTAGGTATGATTTAAATTCTGTTTTATATCACAC | SEQ ID NO: 60 |
| R2866 | GCAAGCCAAGTAACAGTAATGTTTAATTAGGTATGATTTAAATTCTGTTTTATATCACAC | SEQ ID NO: 61 |
| | ************************************************************ | |
| 86028 | TAGCAATGTGGGTTTCTTGTATTGGTATTAACTAAATTACGCATTAATAAAGCGTAATTT | |
| R2846 | TAGCAATGTGGGTTTCTTGTATTGGTATTAACTAAATTACGCATTAATAAAGCGTAATTT | |
| NT36 | TAGCAATGTGGGTTTCTTGTATTGGTATTAACTAAATTACGCATTAATAAAGCGTAATTT | |
| EAGAN | TAGCAATGCGGGTTTCTTGTATTGGTATTAACTAAATTACGCATTAATAAAGCGTAATTT | |
| HK707 | TAGCAATGCGGGTTTCTTGTATTGGTATTAACTAAATTACGCATTAATAAAGCGTAATTT | |
| R2866 | TAGAAATGAGGATTTCTTGTATTGGTATTAACTAAATTACGCATTAATAAGGCGTAATTT | |
| | *** **** ** ************************************** ********* | |
| 86028 | AAGTTAATATCTTGTGGTACATTTAAGAATACAAAATGCCCATCACCTAGTG | |
| R2846 | AAGTTAATATCTTGTGGTACATTTAAGAATACAAAATGCCCATCACCTAGTG | |
| NT36 | AAGTTAATATCTTGTGGTACATTTAAGAATACAAAATGCCCATCGCCTAGTG | |
| EAGAN | AAGTTAATATCTTGTGGTACATTTAAGAATACAAAATGCCCATCGCCTAGTG | |
| HK707 | AAGTTAATATCTTGTGGTACATTTAAGAATACAAAATGCCCATCGCCTAGTG | |
| R2866 | AAGTTAATATCTTGTGGCACATTTAAGAATACAAAATGCCCATCGCCTAGTG | |
| | ***************** ************************** ******* | |

The EAGAN and HK707 sequences are from type b Hi strains; the other four are from NTHi strains. The TAA stop codon of the upstream gene (HI0422) is underlined, as is the reverse complement of the TAG stop codon of the downstream gene (HI0419). The HadA gene is seen between these two sequences, and the key intergenic sequence is SEQ ID NO: 62.

Although H.influenzae strains Rd and F1947 lack the HadA gene, the sequence between HI0419 and HI0422 is longer, and includes a sequence that has homology to the region upstream of and including the first five codons of HadA. The Hi biogroup aegyptius sequences are as follows:

| | |
|---|---|
| CCGACGCAAGCCAAGTAATAGTAATATTTAATTAGGTATGATGTAAATTCTGCTTGAGGC | |
| end of HI0422 * similar to SEQ ID NO: 62 | |
| AAATTTTACATAGGAAATTTTTCTATATTGCTTTAACGTTTTTTTATAGTAGAAGTATAT | |
| ACTCAGTTATGGTTATGGTTACATAGTATAGTTTTACTTTGTTCTAGTTCACTTTAATAA | |
| CCTTAAATAATTGAGGATTTCTTATGAAAAGAAATTTATTAAAACAATCTGTAATCGCTG | SEQ ID NO: 64 |
| M K R N L L K Q S V I A V HadA | SEQ ID NO: 1 |
| TGTTGATAGGTGGCACTACTGTTTCTAATTATGCTTTAGCACAAGCACAAGCACAAGCAC . . . | |
| L I G G T T V S N Y A L A Q A Q A Q A Q . . . | |

The underlined 77mer (SEQ ID NO:63) is also seen in strains Rd and F1947, downstream of HI0422:

SEQ ID NO: 65

AGGATACGAAAAATATCGGCAAACGACGCAAGCCAAGTAACAGTAATGTT
TAGGCTTGTATAGTATAGCTTTGCTTTGTTCTAGTTCAATTTAATAATCT
TAAATAATTAAGGATTTCTTATGAAAAAAAATTTATAGGCTTCGTTTCGC
ACACTCGTTGCTAGTATAGATATGTGAATA . . .

This shared sequence could cause some level of cross-reaction in southern blots even though strains may be HadA-negative.

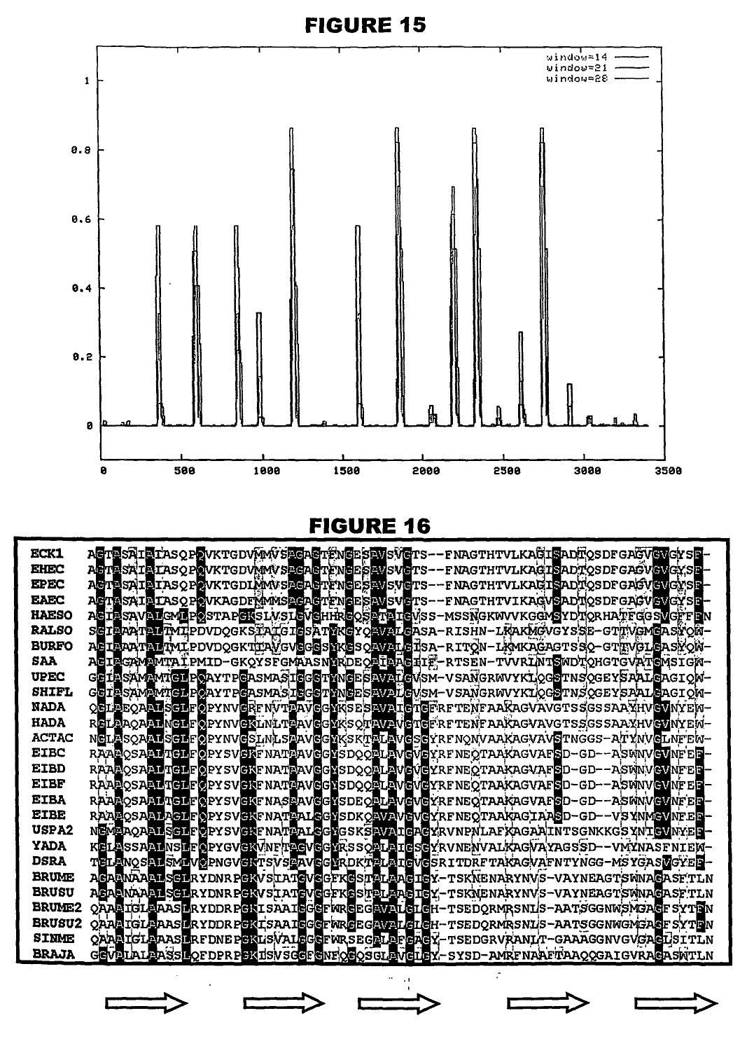

Southern blot experiments on a panel of various Haemophilus strains revealed the presence of HadA in a variety of strains (FIG. 23). All other typeable and non-typeable strains of H.influenzae lacked the hadA gene by this analysis.

HadA Expression in E.coli

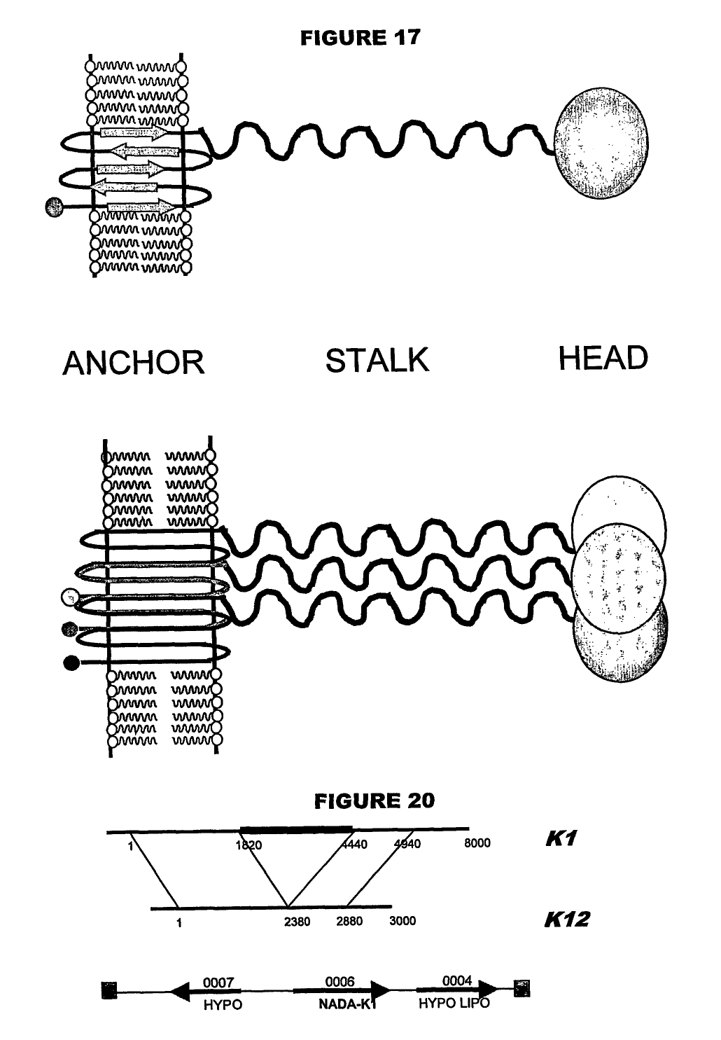

To study the structure and function of HadA, different constructs were prepared for expression in E.coli, as illustrated in FIG. 24: (1) to express the protein as full length (native HadA, or ‘HadA/na’); (2) HadA under the control of NadA leader peptide (‘HadA/LNadA/na’) and (3) as a C-terminal histidine fusion (‘HadA-his’). All the constructs were made in pET21b expression vector and E.coli BL21(DE3) was used as expression host.

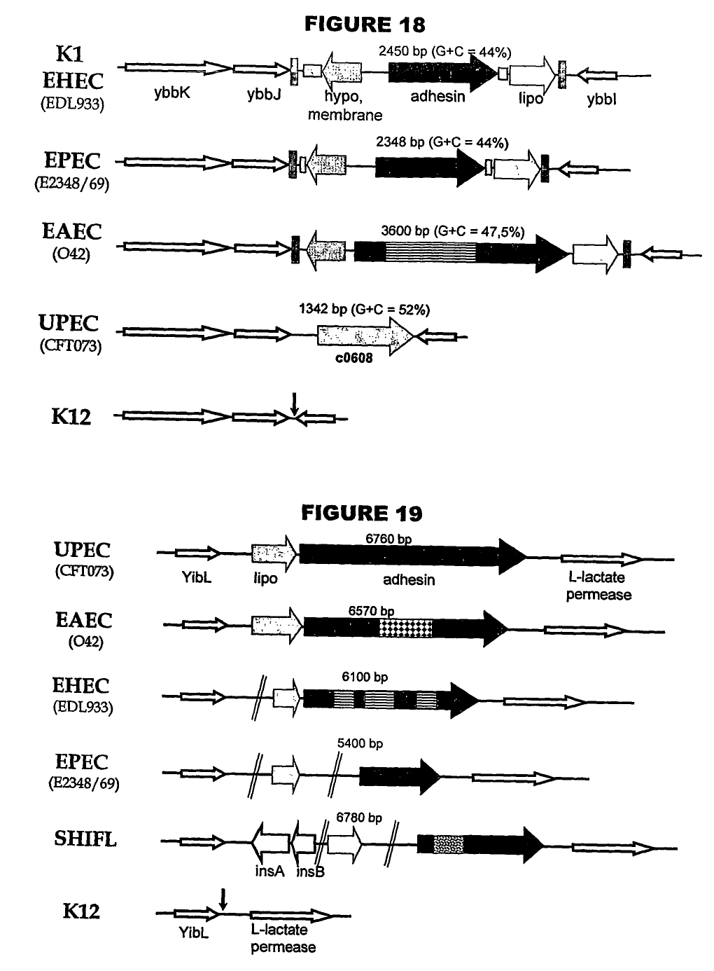

HadA-his was expressed at different temperatures. Total and soluble proteins were analysed by SDS-PAGE (Bis-Tris gels, 12% MOPS; Invitrogen™). The gels are shown in FIG. 25. The soluble protein expressed at 30° C. (lane 9) was purified and was used to immunise mice.

Purification of HadA-His can be performed by the IMAC process, but this was not very efficient for removing all E.coli contaminants. IMAC was thus followed by dialysis in a pH 7.7 buffer, for anionic exchange chromatography. Surprisingly the protein was completely precipitated. Subsequently the protein was dialysed in four different pH condition (6.3, 6.5, 7.7 & 8.5) and precipitation was seen only at pH 7.7. With a theoretical pI of 4.38 then the precipitation should not be isoelectric precipitation.

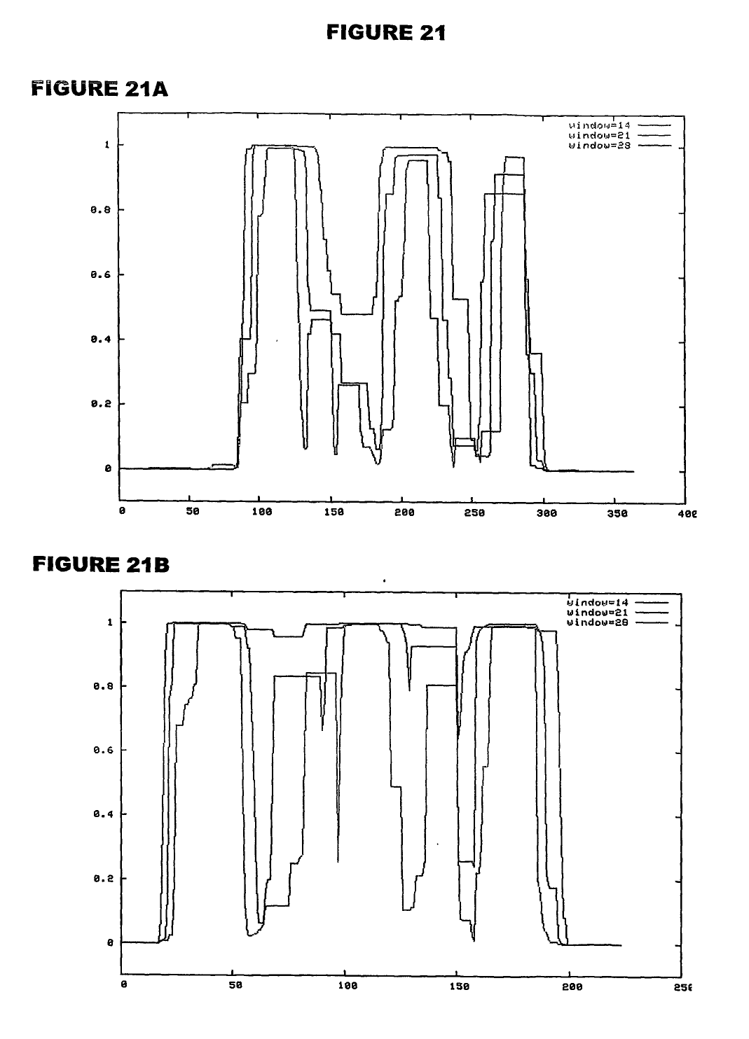

Sera from the mice were used to visualise western blots (12% Mops) of different fractions of E.coli strains and purified recombinant HadA. The first antibody was the anti-HadA (1:1000); the second antibody was anti-mouse immunoglobulin-HRP (DAKO) 1:10000. The results are in FIG. 26.

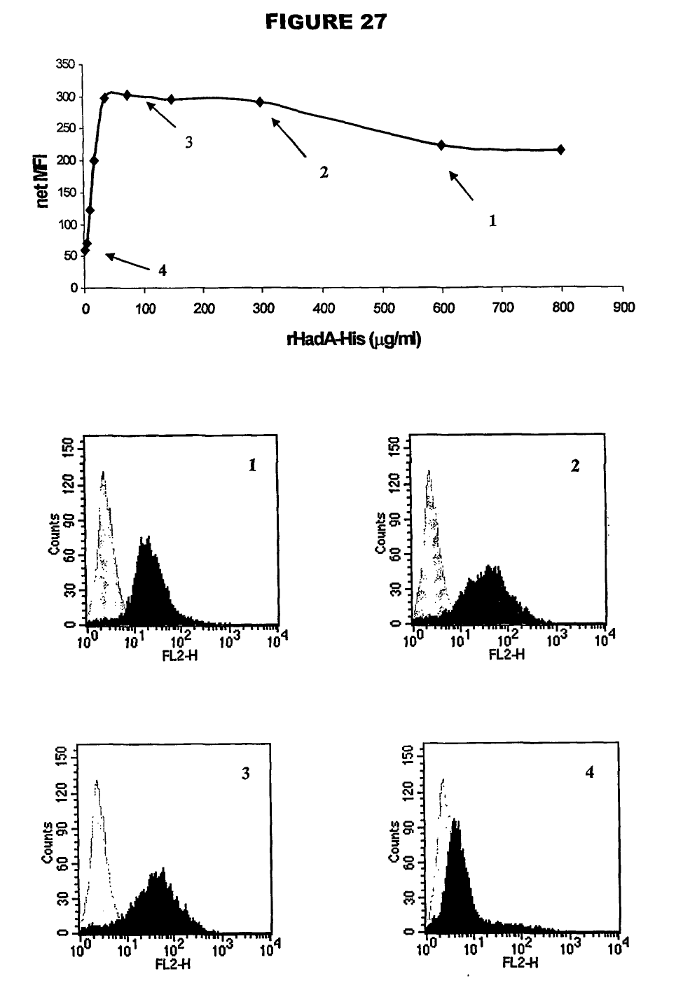

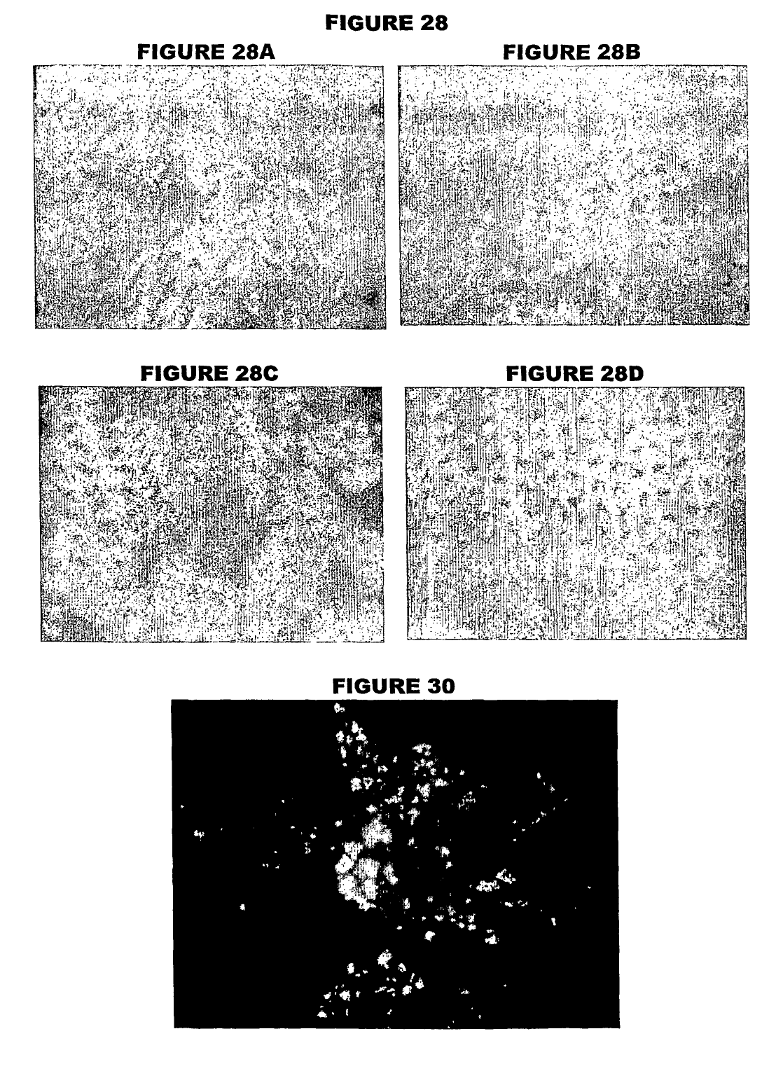

SDS-PAGE of HadA/na and HadA/LNadA/na is shown in FIG. 31. In both overnight and induced cultures, HadA protein was expressed as monomer and as an oligomer (e.g. trimer) that is heat stable in SDS gel. Western blotting is shown in FIG. 32, to confirm the presence of HadA monomer and oligomer in the E.coli bacteria.

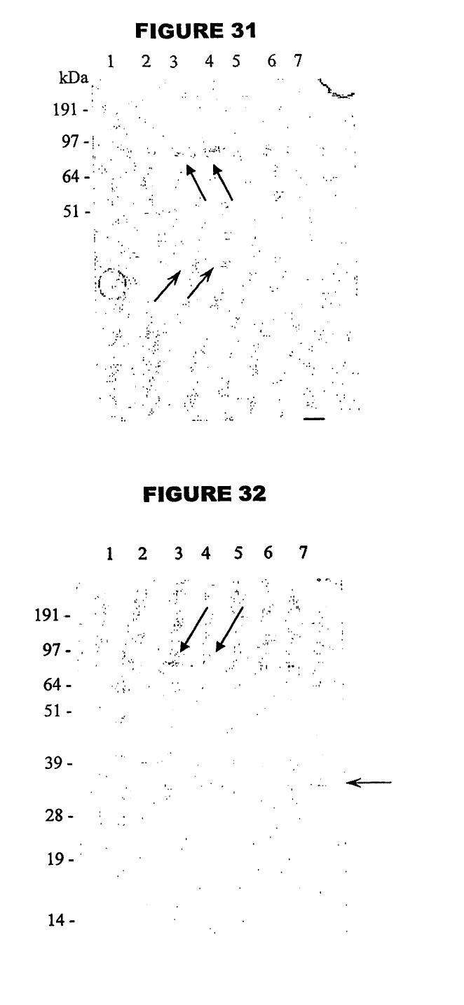

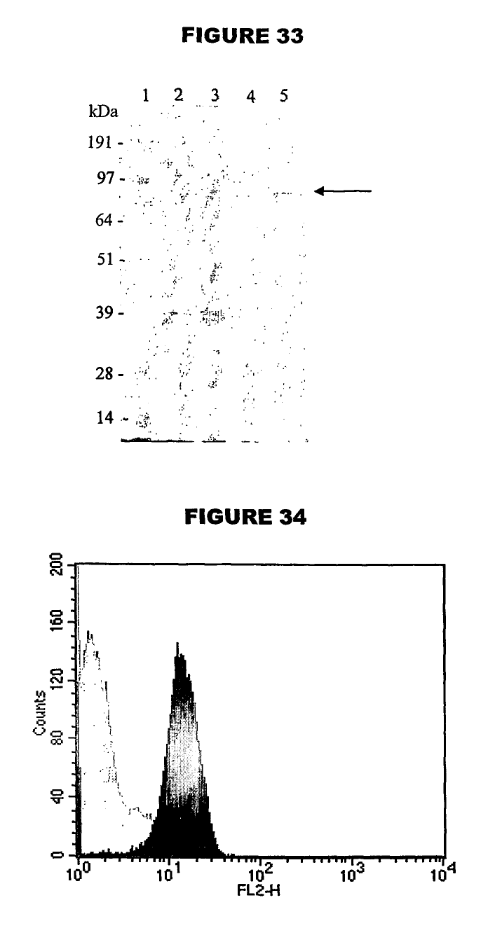

Expression was also studied by examining bacterial outer membrane proteins. FIG. 33 shows SDS-PAGE (Bis-Tris gel 10% MOPS, Invitrogen) of an OMV preparation from E.coli and shows that HadA oligomers are seen in the outer membranes. The cell-surface location was confirmed by FACS, as shown in FIG. 34.

Adhesion

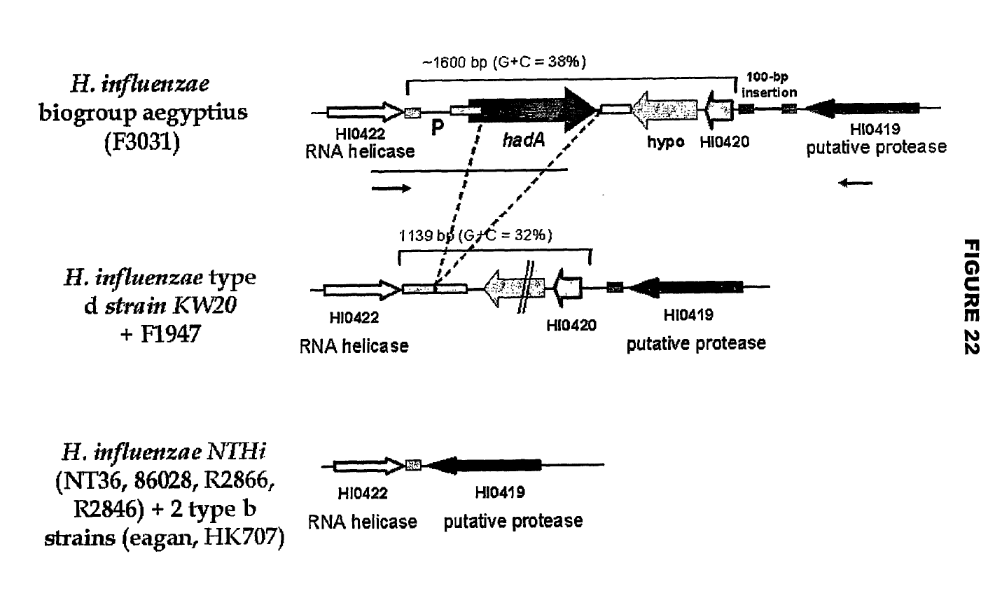

Purified HadA was tested for its ability to bind to Chang epithelial cells. The experiments showed by FACS analysis that HadA-his binds to the cells in a dose dependent manner (FIG. 27).

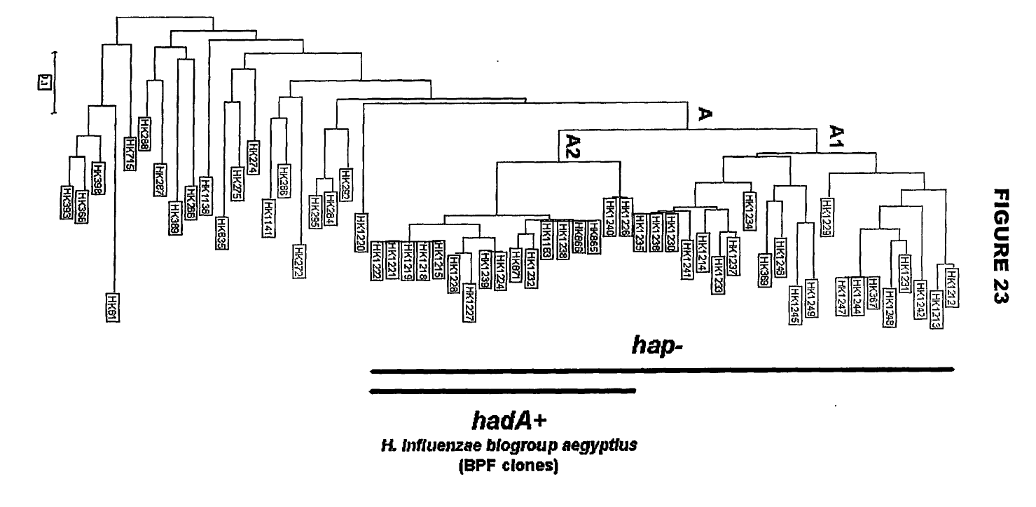

E.coli BL21(DE3) that express HadA/na were tested for aggregation. FIG. 28 shows phase contrast micrographs of cellular aggregates collected from late exponential phase cultures that had been left standing at room temperature for 4 hours. Three different samples are shown in 28A to 28C. The bacteria form visible bacterial “clouds”, and bacterial aggregation can be correlated with microcolony formation. In contrast, cells transformed with only pET plasmid show no aggregates.

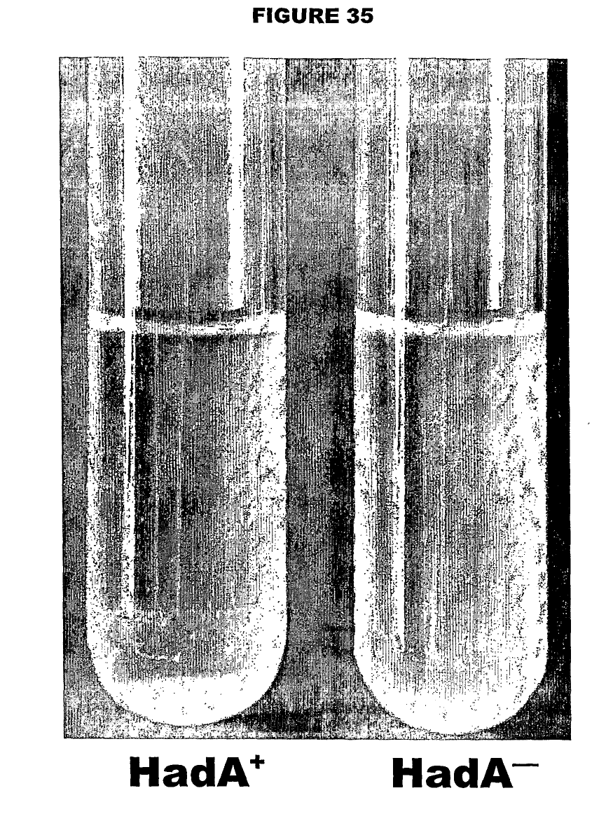

Aggregation was also studied using a tube settling assay. Cultures of E.coli, transformed with either empty pET or HadA/na-containing pET, were incubated to late exponential phase and were then allowed to settle for 4 hours at room temperature. The HadA-expressing bacteria lost turbidity, but the control cells did not (FIG. 35), indicating that HadA promotes bacterial aggregation.

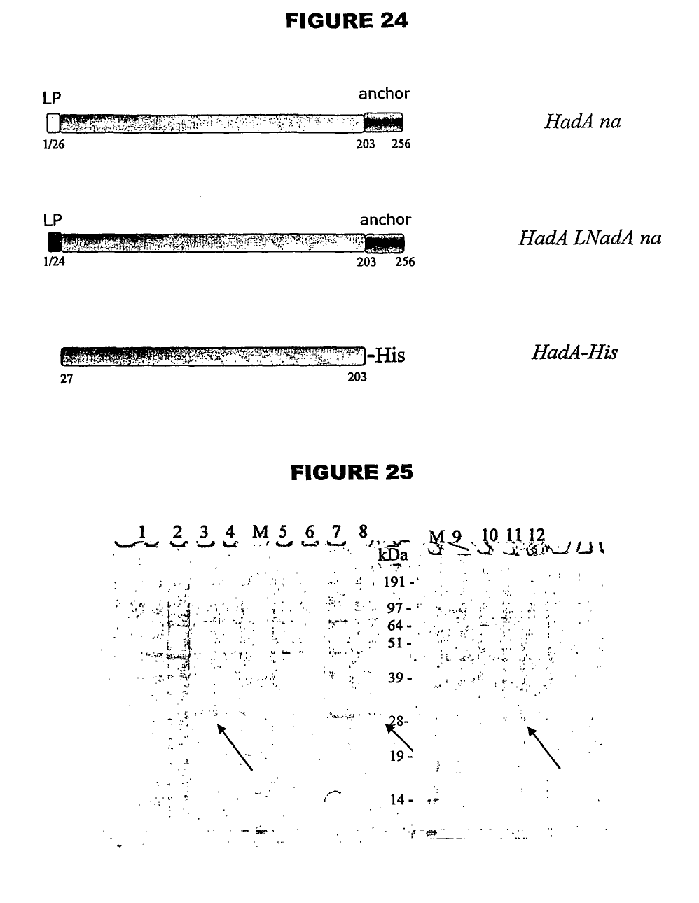

Adhesion and invasion experiments were also performed with E.coli expressing HadA/na and a monolayer of Chang cells. Adherence (invasion) was calculated by counting the number of adherent (invaded) bacteria on cell monolayers (MOI=1:1000). Results were taken as the mean±standard error of the mean of measurements made in triplicate, and are shown in FIG. 29. The numbers of cells showing adhesin and invasion were as follows:

| | Adherent | Invasive |
|---|---|---|
| HadA/na | 1250 ± 344 × 10⁴ | 26.3 ± 6.6 × 10⁴ |
| Empty plasmid | 10.5 ± 2.1 × 10⁴ | 1.5 ± 0.9 × 10⁴ |

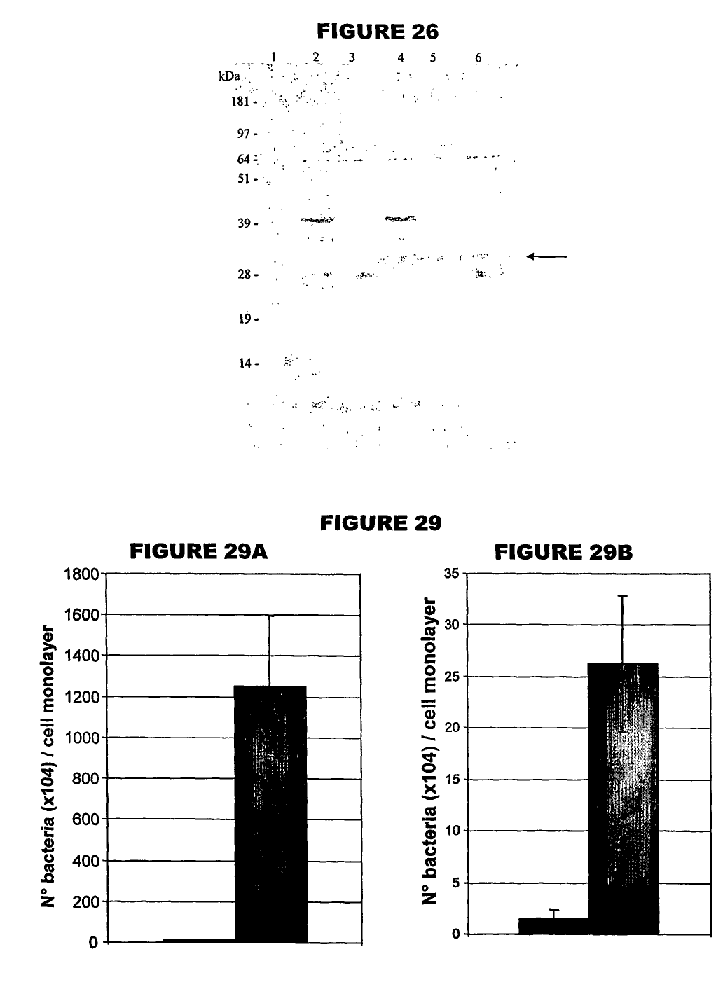

Adhesion and invasion were confirmed by immunofluorescence microscopy analysis (FIG. 30). Extracellular bacteria (green) and intracellular bacteria (red) can be seen.

Further studies of HadA include: construction of an isogenic HadA knockout of BPF Haemophilus influenzae strains for testing in adhesin/invasion assays; testing such knockout mutants to see if adhesin can be complemented by a NadA knockin; competition experiments with HadA and NadA in adhesin/invasion to human cells to see if HadA and NadA bind the same receptor.

It will be understood that the invention has been described by way of example only and modifications may be made whilst remaining within the scope and spirit of the invention.

### REFERENCES (THE CONTENTS OF WHICH ARE HEREBY INCORPORATED BY REFERENCE)

{1} Comanducci et al. (2002) J Exp Med 195:1445-1454.

{2} WO93/13202.

{3} Rappuoli & Pizza, Chapter 1 of Sourcebook of Bacterial Protein Toxins (ISBN 0-12-053078-3).

{4} Pizza et al. (2001) Vaccine 19:2534-41.

{5} Alape-Giron et al. (2000) Eur J Biochem 267:5191-5197.

{6} Kitten et al. (2000) Infect Immun 68:4441-4451.

{7} Gubba et al. (2000) Infect Immun 68:3716-3719.

{8} Boulnois et al. (1991) Mol Microbiol 5:2611-2616.

{9} Breedveld (2000) Lancet 355(9205):735-740.

{10} Gorman & Clark (1990) Semin. Immunol. 2:457-466

{11} Jones et al., Nature 321:522-525 (1986)

{12} Morrison et al., Proc. Natl. Acad. Sci, U.S.A., 81:6851-6855 (1984)

{13} Morrison and Oi, Adv. Immunol., 44:65-92 (1988)

{14} Verhoeyer et al., Science 239:1534-1536 (1988)

{15} Padlan, Molec. Immun. 28:489-498 (1991)

{16} Padlan, Molec. Immunol. 31(3):169-217 (1994).

{17} Kettleborough, C. A. et al., Protein Eng. 4(7):773-83 (1991).

{18} WO99/27961.

{19} WO02/074244.

{20} WO02/064162.

{21} WO03/028760.

{22} Gennaro (2000) Remington: The Science and Practice of Pharmacy. 20th ed., ISBN: 0683306472.

{23} Vaccine design: the subunit and adjuvant approach (1995) Powell & Newman. ISBN 0-306-44867-X.

{24} WO90/14837.

{25} WO00/07621.

{26} WO00/62800.

{27} WO99/27960.

{28} European patent applications 0835318, 0735898 and 0761231.

{29} WO99/52549.

{30} WO01/21207.

{31} WO01/21152.

{32} WO00/23105.

{33} WO99/11241.

{34} WO98/57659.

{35} Del Giudice et al. (1998) Molecular Aspects of Medicine, vol. 19, number 1.

{36} Johnson et al. (1999) Bioorg Med Chem Lett 9:2273-2278.

{37} International patent application WO00/50078.

{38} Singh et al. (2001) J. Cont. Rele. 70:267-276.

{39} Costantino et al. (1992) Vaccine 10:691-698.

{40} Costantino et al. (1999) Vaccine 17:1251-1263.

{41} WO03/007985.

{42} Covacci & Rappuoli (2000) J. Exp. Med. 19:587-592.

{43} WO93/18150.

{44} Covacci et al. (1993) Proc. Natl. Acad. Sci. USA 90: 5791-5795.

{45} Tummuru et al. (1994) Infect. Immun. 61:1799-1809.

{46} Marchetti et al. (1998) Vaccine 16:33-37.

{47} Telford et al. (1994) J. Exp. Med. 179:1653-1658.

{48} Evans et al. (1995) Gene 153:123-127.

{49} WO96/01272 & WO96/01273, especially SEQ ID NO:6.

{50} WO97/25429.

{51} WO98/04702.

{52} Watson (2000) Pediatr Infect Dis J 19:331-332.

{53} Rubin (2000) Pediatr Clin North Am 47:269-285, v.

{54} Jedrzejas (2001) Microbiol Mol Biol Rev 65:187-207.

{55} WO02/077021.

{56} Bell (2000) Pediatr Infect Dis J 19:1187-1188.

{57} Iwarson (1995) AP MIS 103:321-326.

{58} Gerlich et al. (1990) Vaccine 8 Suppl:S63-68 & 79-80.

{59} Hsu et al. (1999) Clin Liver Dis 3:901-915.

{60} Vaccines (1988) eds. Plotkin & Mortimer. ISBN 0-7216-1946-0.

{61} Del Guidice et al. (1998) Molecular Aspects of Medicine 19:1-70.

{62} Gustafsson et al. (1996) N. Engl. J. Med. 334:349-355.

{63} Rappuoli et al. (1991) TIBTECH9:232-238.

{64} Sutter et al. (2000) Pediatr Clin North Am 47:287-308.

{65} Zimmerman & Spann (1999) Am Fam Physician 59:113-118, 125-126.

{66} WO99/24578.

{67} WO99/36544.

{68} WO99/57280.

{69} WO00/22430.

{70} WO00/66791.

{71} WO03/020756.

{72} WO01/64920.

{73} WO01/64922.

{74} Tettelin et al. (2000) Science 287:1809-1815.

{75} Pizza et al. (2000) Science 287:1816-1820.

{76} UK patent application 0227346.4.

{77} UK patent applications 0223741.0, 0305831.0 & 0309115.4.

{78} Bjune et al. (1991) Lancet 338(8775):1093-96

{79} WO01/52885.

{80} Fukasawa et al. (1999) Vaccine 17:2951-2958.

{81} Rosenqvist et al. (1998) Dev. Biol. Stand. 92:323-333.

{82} WO02/02606.

{83} Kalman et al. (1999) Nature Genetics 21:385-389.

{84} Read et al. (2000) Nucleic Acids Res 28:1397-406.

{85} Shirai et al. (2000) J. Infect. Dis. 181(Suppl 3):S524-S527.

{86} WO99/27105.

{87} WO00/27994.

{88} WO00/37494.

{89} WO99/28475.

{90} Ross et al. (2001) Vaccine 19:4135-4142.

{91} Dreesen (1997) Vaccine 15 Suppl:S2-6.

{92} MMWR Morb Mortal Wkly Rep 1998 January 16; 47(1):12, 19.

{93} WO99/24578.

{94} WO99/36544.

{95} WO99/57280.

{96} WO02/079243.

{97} Anderson (2000) Vaccine 19 Suppl 1:S59-65.

{98} Kahn (2000) Curr Opin Pediatr 12:257-262.

{99} Crowe (1995) Vaccine 13:415-421.

{100} McMichael (2000) Vaccine 19 Suppl 1:S101-107.

{101} WO02/34771.

{102} Dale (1999) Infect Dis Clin North Am 13:227-43, viii.

{103} Ferretti et al. (2001) PNAS USA 98: 4658-4663.

{104} WO02/34771.

{105} Kuroda et al. (2001) Lancet 357(9264): 1225-1240; see also pages 1218-1219.

{106} J Toxicol Clin Toxicol (2001) 39:85-100.

{107} Demicheli et al. (1998) Vaccine 16:880-884.

{108} Stepanov et al. (1996) J Biotechnol 44:155-160.

{109} Rosenberg (2001) Nature 411:380-384.

{110} Moingeon (2001) Vaccine 19:1305-1326.

{111} Ramsay et al. (2001) Lancet 357(9251):195-196.

{112} Lindberg (1999) Vaccine 17 Suppl 2:S28-36.

{113} Buttery & Moxon (2000) J R Coll Physicians Lond 34:163-168.

{114} Ahmad & Chapnick (1999) Infect Dis Clin North Am 13:113-133, vii.

{115} Goldblatt (1998) J. Med. Microbiol. 47:563-567.

{116} European patent 0 477 508.

{117} U.S. Pat. No. 5,306,492.

{118} International patent application WO98/42721.

{119} Conjugate Vaccines (eds. Cruse et al.) ISBN 3805549326, particularly vol. 10:48-114.

{120} Hermanson (1996) Bioconjugate Techniques ISBN: 0123423368 or 012342335X.

{121} Research Disclosure, 453077 (January 2002)

{122} EP-A-0372501

{123} EP-A-0378881

{124} EP-A-0427347

{125} WO93/17712

{126} WO94/03208

{127} WO98/58668

{128} EP-A-0471177

{129} WO00/56360

{130} WO91/01146

{131} WO00/61761

{132} WO01/72337

{133} Robinson & Torres (1997) Seminars in Immunology 9:271-283.

{134} Donnelly et al. (1997) Annu Rev Immunol 15:617-648.

{135} Scott-Taylor & Dalgleish (2000) Expert Opin Investig Drugs 9:471-480.

{136} Apostolopoulos & Plebanski (2000) Curr Opin Mol Ther 2:441-447.

{137} Ilan (1999) Curr Opin Mol Ther 1:116-120.

{138} Dubensky et al. (2000) Mol Med 6:723-732.

{139} Robinson & Pertmer (2000) Adv Virus Res 55:1-74.

{140} Donnelly et al. (2000) Am J Respir Crit Care Med 162(4 Pt 2):S190-193.

{141} Davis (1999) Mt. Sinai J. Med. 66:84-90.

{142} Piddock (1998) Curr Opin Microbiol 1:502-8.

{143} Nielsen (2001) Expert Opin Investig Drugs 10:331-41.

{144} Good & Nielsen (1998) Nature Biotechnol 16:355-358.

{145} Rahman et al. (1991) Antisense Res Dev 1:319-327.

{146} Methods in Enzymology volumes 313 & 314.

{147} Manual of Antisense Methodology (eds. Hartmann & Endres).

{148} Antisense Therapeutics (ed. Agrawal).

{149} WO99/02673.

{150} WO99/13893.

{151} Vidal & Endoh (1999) TIBTECH 17:374-381.

{152} Current Protocols in Molecular Biology (F. M. Ausubel et al., eds., 1987) Supplement 30.

{153} Smith & Waterman (1981) Adv. Appl. Math. 2: 482-489.

{154} Hoiczyk et al. (2000) EMBO J 19:5989-5999.

{155} Paton et al. (2001) Infect Immun 69:6999-7009.

{156} Smoot et al. (2002) Infect Immun 70:2694-2699.

{157} Fleischmann et al. (1995) Science 269:538-540.

1. A polypeptide comprising one or more of: (a) an amino acid sequence selected from the group consisting of SEQ ID NOS: 51, 1, 2, 3, 4, 5, 6, 7, 8, 9, 10, 11, 12, 13, 14, 15, 16, 17, 18, and 54; (b) an amino acid sequence having at least 70% identity to a sequence as defined in (a); and/or (c) an amino acid sequence comprising a fragment of at least 8 consecutive amino acids of a sequence as defined in (a).

2. The polypeptide of claim 1, wherein the fragment of (c) does not include one or more of four domains of the sequence of (a).

3. The polypeptide of claim 1, wherein the fragment of (c) includes at least one complete domain of the sequence of (a).

4. The polypeptide of claim 1, in oligomeric form.

5. A polypeptide of the formula NH2 A-{-X-L-}X-B—COOH, wherein: X comprises an amino acid sequence: (a) having at least 70% identity to one or more of SEQ ID NOS: 1-18, 51 or 54; and/or (b) which is a fragment of at least 8 consecutive amino acids of one or more of SEQ ID NOS: 1-18, 51 or 54; L is an optional linker amino acid sequence; A is an optional N terminal amino acid sequence; B is an optional C terminal amino acid sequence; and x is 1, 2, 3, 4, 5, 6, 7, 8, 9, 10, 11, 12, 13, 14, 15, 16, 17, 18, 19 or 20.

6. A polypeptide comprising the amino acid sequence -A-W₁—W₂—W₃—W₄—B—, wherein:
A is an optional N-terminus sequence;
B is an optional C-terminus sequence;
W₁ is an optional amino acid sequence: (a) having at least 70% identity to the leader peptide of one or more of SEQ ID NOS: 1-18 or 51; and/or (b) which is a fragment of at least 8 consecutive amino acids of the leader peptide of one or more of SEQ ID NOS: 1-18 or 51; W₂ is an optional amino acid sequence: (a) having at least 70% identity to the globular head of one or more of SEQ ID NOS: 1-18 or 51; and/or (b) which is a fragment of at least 8 consecutive amino acids of the leader peptide of one or more of SEQ ID NOS: 1-18 or 51; W₃ is an optional amino acid sequence: (a) having at least 70% identity to the coiled-coil domain of one or more of SEQ ID NOS: 1-18 or 51; and/or (b) which is a fragment of at least 8 consecutive amino acids of the leader peptide of one or more of SEQ ID NOS: 1-18 or 51; W₄ is an optional amino acid sequence: (a) having at least 70% identity to the transmembrane anchor region of one or more of SEQ ID NOS: 1-18 or 51; and/or (b) which is a fragment of at least 8 consecutive amino acids of the leader peptide of one or more of SEQ ID NOS: 1-18 or 51;
provided that at least one of W₁, W₂, W₃ or W₄ is present.

7. An adhesin from Haemophilus aeyptius, wherein the adhesin comprises: (a) amino acid sequence SEQ ID NO: 52; (b) an amino acid sequence having at least 70% identity to SEQ ID NO: 52, and/or (c) an amino acid sequence which is a fragment of at least 8 consecutive amino acids of SEQ ID NO: 52.

8. An antibody that binds to the polypeptide of claim 1.

9. Nucleic acid encoding the polypeptide of claim 1.

10. A pharmaceutical composition comprising the polypeptide of claim 1.

11. (canceled)

12. (canceled)

13. A method for raising an immune response in a mammal comprising the step of administering an effective amount of the composition of claim 10 to said mammal.

14. Nucleic acid encoding the antibody of claim 8.

15. A pharmaceutical composition comprising the antibody of claim 8.

16. A pharmaceutical composition comprising the nucleic acid of claim 9.

17. A pharmaceutical composition comprising the nucleic acid of claim 14.

18. A pharmaceutical composition comprising the polypeptide of claim 5.

19. A pharmaceutical composition comprising the polypeptide of claim 6.

20. A pharmaceutical composition comprising the polypeptide of claim 7.

21. A method for raising an immune response in a mammal comprising the step of administering an effective amount of the composition of claim 18 to said mammal.

22. A method for raising an immune response in a mammal comprising the step of administering an effective amount of the composition of claim 19 to said mammal.

23. A method for raising an immune response in a mammal comprising the step of administering an effective amount of the composition of claim 20 to said mammal.
